(19) United States
(12) Patent Application Publication
(10) Pub. No.: US 2024/0165116 A1
(43) Pub. Date: May 23, 2024

(54) ROCK2 INHIBITOR FOR THE TREATMENT OF VIRAL INFECTION

(71) Applicant: Graviton Bioscience B.V., Amsterdam (NL)

(72) Inventors: Samuel D. Waksal, New York, NY (US); Rui Wu, Wilmington, DE (US)

(21) Appl. No.: 18/547,257

(22) PCT Filed: Feb. 22, 2022
(86) PCT No.: PCT/US2022/017312

§ 371 Date: Aug. 21, 2023

Related U.S. Application Data
(60) Provisional application No. 63/152,260, filed on Feb. 22, 2021.

(51) Int. Cl. A61K 31/506; A61P 11/00; A61P 31/14

(52) U.S. Cl. CPC A61K 31/506; A61P 11/00; A61P 31/14

(57) ABSTRACT

The disclosure provides compositions and methods comprising selective inhibitors of Rho-associated coiled-coil kinase 2 (ROCK2) for use in the treatment of viral infections, particularly coro-navirus infections such as SARS-CoV-2, and in the treatment of seguelae resulting from the viral infection, including sequelae resulting from coronavirus infection.

### FIELD

The present disclosure relates to methods and compositions for the treatment of viral infections using Rho-associated coiled-coil kinase (ROCK) inhibitors, and particularly inhibitors of Rho-associated coiled-coil kinase 2 (ROCK2).

### BACKGROUND

Viral infections are common among animals and humans. For example, the COVID-19 pandemic, caused by the SARS-CoV-2 virus, has threatened public health all over the world. Strikingly, this pandemic has resulted in >100 million people infected worldwide, with >2 million people killed by the end of 2020.

Coronaviruses are a large family of enveloped viruses with a positive-sense, single-stranded RNA genome and belong to the Coronaviridae family, Nidovirales order. Coronavirus infections are concentrated mainly in the upper respiratory system and gastrointestinal tract, although the lower respiratory system may be involved in more serious infections. According to specific virus and host cell types, the symptoms and pathological damage caused by coronavirus infection may be quite different. Some coronaviruses, including HCoV-NL63, HCoV-229E, and HCoV-0C43, continually circulate in the human population and produce mild symptoms similar to the common cold. Other coronaviruses may cause severe respiratory illness with high morbidity and mortality, including Severe Acute Respiratory Syndrome coronavirus (SARS-CoV-1), Middle East Respiratory Syndrome coronavirus (MERS-CoV), and SARS-CoV-2. MERS-CoV was first reported in Saudi Arabia in 2012 and spread to several other countries. SARS-CoV-1 was first recognized in China in 2002 and led to a worldwide outbreak in 2002 and 2003. Other human coronaviruses include 222E (alpha coronavirus), NL63 (alpha coronavirus), OC43 (beta coronavirus), and HKU1 (beta coronavirus).

Persistent and diverse post-viral symptoms have been described in survivors of coronavirus infection, including survivors of Covid-19, even in those with a mild initial disease course. Currently, no curative treatments are available for post-viral syndromes and therapy is directed at symptom alleviation and coping strategies. Additionally, the economic effect of post-viral syndromes can be substantial, including loss of productivity and employment, and increased need for disability benefits and financial support. There is a substantial and unmet need for therapies to treat coronavirus infections, and to treat the sequelae resulting from coronavirus infection.

Rho-associated coiled-coil kinase (ROCK) is a serine/threonine kinase from the AGC (PKA, PKG, and PKC) kinase family and comprises two isoforms, ROCK1 and ROCK2. The two isoforms are expressed and regulated differently in specific tissues. For example, ROCK1 is ubiquitously expressed at a relatively high level, while ROCK2 is preferentially expressed in certain tissues including heart, brain and skeletal muscle. ROCK is a target of the small GTPase Rho and is involved in diverse cellular activities achieved by phosphorylating downstream effector proteins (MLC, LIMK, ERM, MARCKS, CRMP-2, etc.). Studies have shown that various diseases (e.g., pulmonary fibrosis, cardiac-cerebral vascular disease, neurological disease and cancer etc.) are related to the pathways mediated by ROCK. As such, ROCK has been considered as an important target in the development of novel drugs.

The present disclosure relates to the previous unrecognized and surprising potent anti-viral effects of ROCK2 inhibitors and their use for the treatment of viral infections, including coronavirus infections such as a SARS-CoV-1 infection, a SARS-CoV-2 infection, or a MERS-CoV infection. Additionally, ROCK2 inhibition treats many of the secondary conditions that may be result from the viral infection, including inflammation, fibrosis, and cytokine storm.

### SUMMARY

In one aspect, the disclosure provides antiviral compositions comprising a Rho-associated coiled-coil kinase (ROCK2) inhibitor. In one aspect, the disclosure provides methods of treating a subject by administering to the subject a therapeutically effective amount of a ROCK2 inhibitor, or a composition comprising a ROCK2 inhibitor. Such methods and compositions described herein may be useful for the treatment of an individual afflicted with or suspected of being afflicted with a viral infection. In embodiments, the viral infection is a coronavirus infection. In embodiments, the viral infection is a SARS-CoV-1 infection, a SARS-CoV-2 infection, or a MERS-CoV infection. In embodiments, the viral infection is caused by SARS-CoV-2 variant Delta and/or SARS-CoV-2 variant Omicron. In embodiments, the methods and compositions described herein are useful for the treatment and prevention of sequelae resulting from the viral infection, including sequelae resulting from coronavirus infection. In embodiments, the methods and compositions described herein are useful for the treatment and prevention of sequelae resulting from a SARS-CoV-1 infection, a SARS-CoV-2 infection, or a MERS-CoV infection. In embodiments, the viral infection is caused by SARS-CoV-2 variant Delta and/or SARS-CoV-2 variant Omicron. In embodiments, the sequelae include one or more of the group consisting of fatigue, dyspnea (difficulty breathing), cough, arthralgia (joint pain), myalgia, headache, chest pain, fever, palpitations, myocardial inflammation, ventricular dysfunction, stroke, pulmonary function abnormalities, fibrosis (such as pulmonary fibrosis), renal dysfunction rash, alopecia, olfactory and/or gustatory dysfunction, sleep dysregulation, cognitive impairment altered, memory impairment, depression, anxiety, changes in mood and combinations thereof. In embodiments, the sequelae includes fibrosis. In one aspect, the disclosure provides a method of treating a viral infection in a subject in need thereof, the method comprising administering to the human subject a therapeutically effective amount of a ROCK2 selective inhibitor, wherein the ROCK2 selective inhibitor is a compound having the Formula I to XII, and particularly (6-(4-((4-(1H-pyrazol-4-yl)phenyl)amino)pyrimidin-2-yl)-1-methyl-1H-indol-2-yl)(3,3-difluoroazetidin-1-yl)methanone. In one aspect, the disclosure provides a method for the treatment and prevention of sequelae resulting from the viral infection in a subject in need thereof, the method comprising administering to the human subject a therapeutically effective amount of a ROCK2 selective inhibitor, wherein the ROCK2 selective inhibitor is a compound having the Formula I to XII, and particularly (6-(4-((4-(1H-pyrazol-4-yl)phenyl)amino)pyrimidin-2-yl)-1-methyl-1H-indol-2-yl)(3,3-difluoroazetidin-1-yl)methanone.

In some embodiments, the method comprises administering to the subject a therapeutically effective amount of at least one other therapeutic agent. The at least one other therapeutic agent may be another antiviral agent, a corticosteroid, an anti-inflammatory signal transduction modulator, a β2-adrenoreceptor agonist bronchodilator, an anticholinergic agent, a mucolytic agent, hypertonic saline, or a mixture thereof.

In one aspect, the disclosure provides a method of treating a viral infection in a subject in need thereof, comprising administering to the human subject a therapeutically effective amount of a ROCK2 selective inhibitor, wherein the ROCK2 selective inhibitor is a compound having the Formula I to XII, and particularly (6-(4-((4-(1H-pyrazol-4-yl)phenyl)amino)pyrimidin-2-yl)-1-methyl-1H-indol-2-yl)(3,3-difluoroazetidin-1-yl)methanone and a therapeutically effective amount of at least one other antiviral agent. The at least one other antiviral agent may be a nucleoside or nucleotide analog, or a pharmaceutically acceptable salt or prodrug thereof.

In embodiments, provided is a method of treating a viral infection in a subject in need thereof or a method of treating and preventing sequelae resulting from the viral infection in a subject in need thereof, the method comprising administering a ROCK2 inhibitor disclosed herein to the patient at a total dose of about 200 mg to about 500 mg per day. In an embodiment, the ROCK2 inhibitor is administered to the patient at a total dose of about 200 mg per day. In an embodiment, the ROCK2 inhibitor is administered to the patient at a total dose of about 300 mg per day. In an embodiment, the ROCK2 inhibitor is administered to the patient at a total dose of about 400 mg per day. In an embodiment, the ROCK2 inhibitor is administered to the patient at a total dose of about 500 mg per day. In an embodiment, the ROCK2 inhibitor is administered in one daily administration. In an embodiment, the ROCK2 inhibitor is administered in two daily administrations. In embodiments, the ROCK2 inhibitor is administered to the patient within about 1, about 2, about 3, about 4, about 5, about 6, about 7, about 8, about 9, about 10, about 11, about 12, about 24, about 28, about 72, about 96, or about 120 hours after the patient was first exposed to the virus causing the viral infection. In embodiments, the ROCK2 inhibitor is administered to the patient within about 1, about 2, about 3, about 4, about 5, about 6, about 7, about 8, about 9, about 10, about 11, about 12, about 24, about 28, about 72, about 96, or about 120 hours after the patient has developed symptoms caused by the viral infection. In embodiments, the ROCK2 inhibitor is administered to the patient for about 1, about 2, about 3, about 4, about 5, about 6, about 7, about 8, about 9, or about 10 weeks after the symptoms have subsided.

### BRIEF DESCRIPTION OF THE DRAWINGS

FIG. 1. Cellular Toxicity: LDH Assay. Immortalized human airway epithelial cells (Calu-3) were treated with the listed concentrations of the Formula XII compound for 2 h at 37° C. followed by challenge with SARS-CoV-2 for 6 h. After washing, the cells were maintained for 48 hours and LDH assay performed.

FIG. 2. Efficacy: SARS-CoV-2 CPN in Supernatant. Immortalized human airway epithelial cells (Calu-3) were treated with the listed concentrations of the Formula XII compound for 2 h at 37° C. followed by challenge with SARS-CoV-2 for 6 h. After washing, the cells were maintained for 48 hours and viral infection was analyzed by qPCR of the supernatant. Each data point represents the mean of three replicates.

FIG. 3. Efficacy: Classical Dose-Response Curve. This figure provides the dose-response curve for the experiment described in Example 1 and FIG. 2. Based on the best-fit values, an EC₅₀ of 124 nM was calculated.

FIG. 4. Efficacy: Median-Effect Model Analysis. This figure provides the median-effect model analysis for the experiment described in Example 1 and FIG. 2. Fa, fraction affected; Fu, fraction unaffected; D, dose (μM)

FIG. 5. Efficacy: Predicted EC50—EC97 Values. This figure provides the predicted EC₅₀ to EC₉₇ values for the experiment described in Example 1 and FIG. 4.

FIGS. 6A, 6B, and 6C. Efficacy: Viral Load in Lung Lysates. This figure shows that Formula XII reduces the spread of SARS-CoV2 in the lungs of mice in vivo. Shown are viral copies/mg of lungs for initial viral loads of 100 pfu SARS-CoV2 (FIG. 6A), 1000 pfu SARS-CoV2 (FIG. 6B), and 10,000 pfu SARS-CoV2 (FIG. 6C). Groups from left to right: Vehicle, Formula XII (300 mg/kg), Remdesivir (RDV). Inserts: Formula XII (300 mg/kg) (left), Remdesivir (RDV) (right).

### DETAILED DESCRIPTION

The compounds, compositions and methods described herein provide selective inhibitors of Rho-associated coiled-coil kinase 2 (ROCK2) for use in the treatment of viral infections, particularly coronavirus infections such as a SARS-CoV-1 infection, a SARS-CoV-2 infection, or a MERS-CoV infection, and in the treatment and prevention of sequelae resulting from the viral infection, including sequelae resulting from coronavirus infection.

### ROCK2 Inhibitors

The compounds for use in the methods and compositions disclosed herein are ROCK inhibitors, and in particular ROCK2 selective inhibitors. The compounds provide excellent inhibitory activity of ROCK (preferably ROCK2), good selectivity (higher selectivity towards ROCK2 as compared with ROCK1), good physicochemical properties (e.g., solubility, physical and/or chemical stability), improved pharmacokinetic properties (e.g., improved bioavailability, proper half-life and duration of action), improved safety (low toxicity and/or less side effects, wide therapeutic window), and the like.

As provided herein, ROCK2 inhibitors have previously unrecognized and surprisingly potent anti-viral effects. Without being bound by theory, ROCK2 inhibitors, such as, for example, compounds of the Formulas I to XII, may interfere with one or more of: (1) pathways used by the virus to enter cells; (2) the cellular cytoskeleton used by the virus as a track for migration and spread; (3) pathways used by the virus to upregulate the energy metabolism of a cell; and (4) pathways used by the virus to spread to other cells. The inhibitors of ROCK2 interfere with viral interaction with cytoskeletons actin filaments, microtubules, and/or intermediate filaments, which are heavily involved in the life cycle and pathological damages caused by viruses, and particularly coronavirus. Additionally, the ROCK2 inhibitors have potent effects against many of the secondary conditions caused by the viral infection, including inflammation, fibrosis, and cytokine and bradykinin storm.

According to one aspect, the present disclosure provides methods of treating a subject by administering to the subject a therapeutically effective amount of a selective ROCK2 inhibitor, or a composition comprising the selective ROCK2 inhibitor, wherein the ROCK2 selective inhibitor has the structure of Formula I:

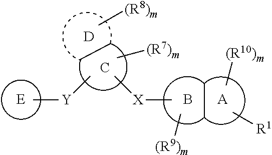

or a pharmaceutically acceptable salt, ester, stereoisomer, polymorph, solvate, N-oxide, or isotopically labeled compound thereof, wherein:
X and Y are each independently selected from the group consisting of a direct bond, C(═O), O, S(═O), and NR;
R is selected from the group consisting of H, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);
ring A and ring B are each independently selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O); provided that when ring B is a heterocycle containing a nitrogen atom, ring B is not attached to X via the nitrogen atom;
ring C is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring D is absent, or is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring E is selected from the group consisting of:

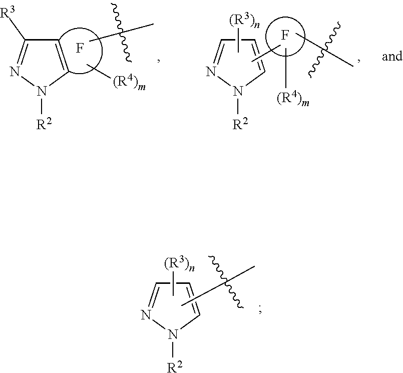

ring F is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
R¹ is selected from the group consisting of H, —NH₂, C₁₋₆ alkyl, C₆₋₁₀ aryl, 5- to 14-membered heteroaryl, N-methylpyrrolidinyl, N-methylpiperidinyl,

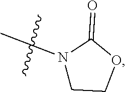 [SMILES: *N1CCOC1=O]

acetyl,

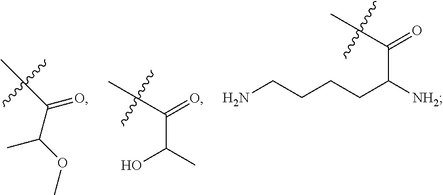

—C(═O)-(C₁₋₆ alkylene)n-CF₃, —C(═O)-(C₁₋₆ alkylene) CN, —C(═O)-(saturated or partially unsaturated C₃₋₁₀ cyclic hydrocarbyl), —NHC(═O)-(saturated or partially unsaturated C₃₋₁₀ cyclic hydrocarbyl), —C(═O)-(saturated or partially unsaturated 3- to 10-membered heterocyclyl), —C(═O)-C₁₋₆ alkylene-(saturated or partially unsaturated 3- to 10-membered heterocyclyl), —C(═O)-(5- to 14-membered heteroaryl), —C(═O)-C₁₋₆ alkylene-NH(C₁₋₆ alkyl), —C(═O)-C₁₋₆ alkylene-N(C₁₋₆ alkyl)₂, N-methylpiperazine substituted acetyl, —S(═O)₂R^{1a}, —P(═O)R^{1a}R^{1b},

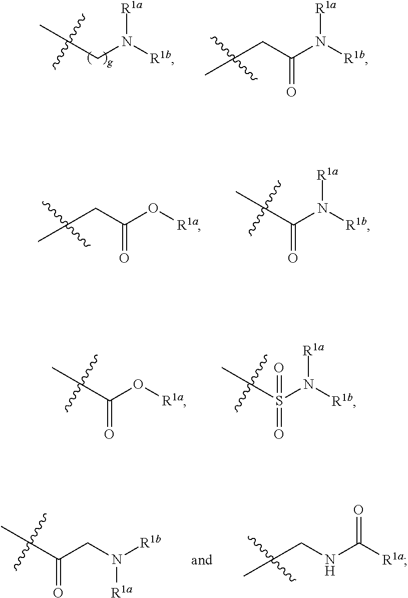

provided that when one of R¹ and R¹⁰ is C₁₋₆ alkyl, and the other is H or C₃₋₁₀ cyclic hydrocarbyl, at least one of X and Y is a direct bond, and ring C is not a 5-membered heteroaromatic ring; when one of R¹ and R¹⁰ is H, and the other is

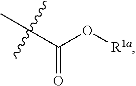

ring C is not a 5-membered heteroaromatic ring; when both R¹ and R¹⁰ are H, ring A contains at least one nitrogen atom, and is not a 5- or 6-membered ring; when one of R¹ and R¹⁰ is H, and the other is

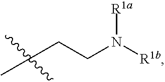

ring C is not a 5-membered heteroaromatic ring; and when one of R¹ and R¹⁰ is H, and the other is H or acetyl, ring D is absent;
R^{1a} and R^{1b} are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, NR⁵—S(═O)₂—R⁶, —NR⁵—C(═O)—NR⁵R⁶, -C₁₋₆ alkylene-NR⁵R⁶, -C₁₋₆ alkylene-OR⁵ and —O-C₁₋₆ alkylene-NR⁵R⁶, provided that when one of R^{1a} and R^{1b} is n-propyl, the other is not H; or R^{1a} and R^{1b} together with the atom to which they are attached form a 3- to 12-membered heterocycle or heteroaromatic ring;
R², R³, R⁴, R⁷, R⁸, R⁹ and R¹⁰, at each occurrence, are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, OC(═O)R⁵, —C(═O)OR⁵, OR⁵, SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂-R⁶, —NR⁵—C(═O)—NR⁵R⁶, -C₁₋₆ alkylene-NR⁵R⁶, -C₁₋₆ alkylene-O(P═O)(OH)₂ and —O-C₁₋₆ alkylene-NR⁵R⁶;
the above alkyl, alkylene, alkenyl, alkynyl, cyclic hydrocarbyl, hydrocarbon ring, heterocyclyl, heterocycle, aryl, aromatic ring, heteroaryl, heteroaromatic ring and aralkyl, at each occurrence, are each optionally substituted with one or more substituents independently selected from the group consisting of halogen, hydroxyl, oxo, amino, cyano, nitro, C₁₋₆ alkyl, C₂₋₆ alkenyl, C₂₋₆ alkynyl, C₃₋₆ cyclic hydrocarbyl, 3- to 10-membered heterocyclyl, C₆₋₁₀ aryl, 5- to 14-membered heteroaryl, C₆₋₁₂ aralkyl, ═N—OR⁵, —C(═NH)NH₂, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂-R⁶, —NR⁵—C(═O)—NR⁵R⁶, -C₁₋₆ alkylene-NR⁵R⁶ and —O-C₁₋₆ alkylene-NR⁵R⁶, and the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, C₁₋₆ alkyl, C₃₋₆ cyclic hydrocarbyl, 3- to 10-membered heterocyclyl, C₆₋₁₀ aryl, 5- to 14-membered heteroaryl and C₆₋₁₂ aralkyl;
R⁵ and R⁶, at each occurrence, are each independently selected from the group consisting of H, alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3;
n is an integer of 0, 1 or 2;
i is an integer of 0, 1 or 2; and
g is an integer of 0, 1, 2, 3 or 4.

In an embodiment, the present disclosure provides a method for treating the viral infections, diseases or conditions disclosed herein using a compound of Formula II to IX:

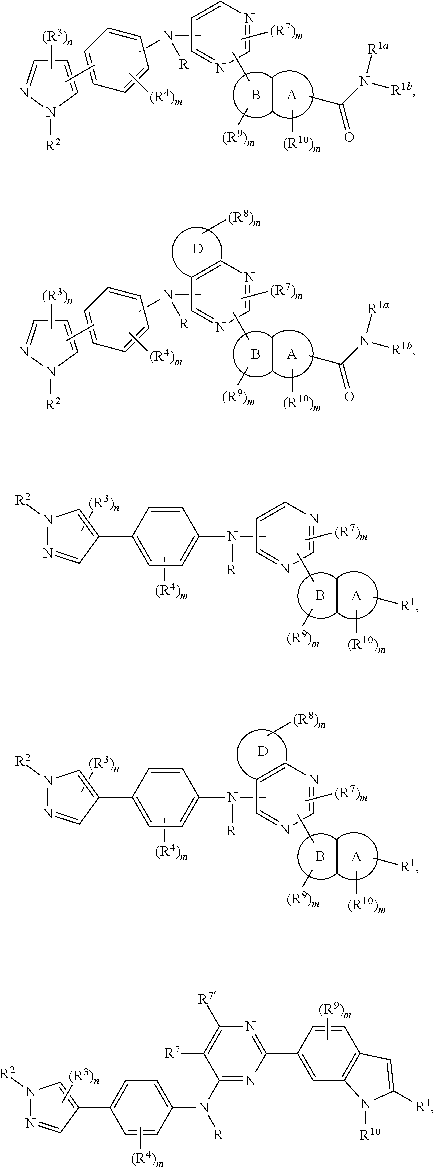

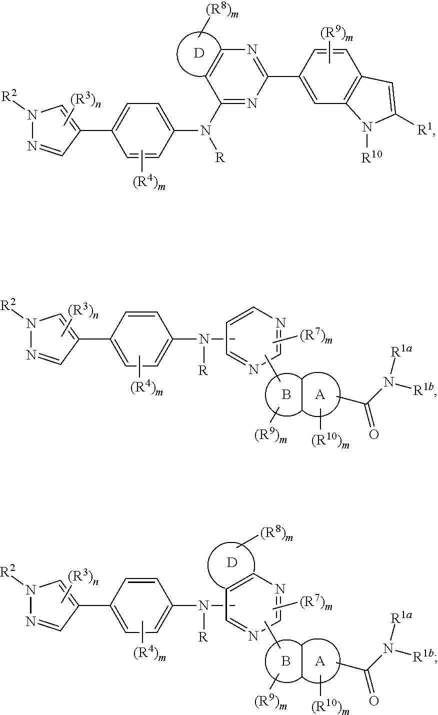

or a pharmaceutically acceptable salt, ester, stereoisomer, polymorph, solvate, N-oxide, or isotopically labeled compound thereof, wherein each of ring A, ring B, ring D, R, R¹, R^{1a}, R^{1b}, R², R³, R⁴, R⁷, R^{7′}, R⁸, R⁹, R¹⁰ n and m are defined above.

In an embodiment, the present disclosure provides a method for treating the viral infections, diseases or conditions disclosed herein using a compound of Formula (X) or Formula (XI):

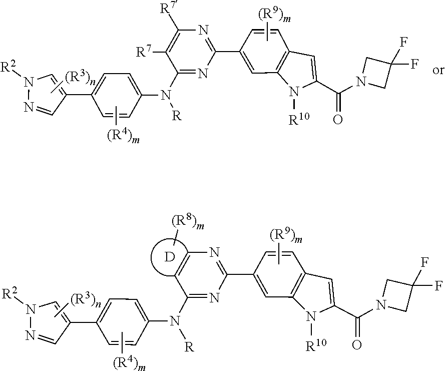

or a pharmaceutically acceptable salt, ester, stereoisomer, polymorph, solvate, N-oxide, or isotopically labeled compound thereof, wherein:
R is selected from the group consisting of H and C₁₋₆ alkyl;
ring D is saturated or partially unsaturated 3- to 10-membered heterocycle, C₆₋₁₀ aryl, or 5- to 10-membered heteroaromatic ring, preferably

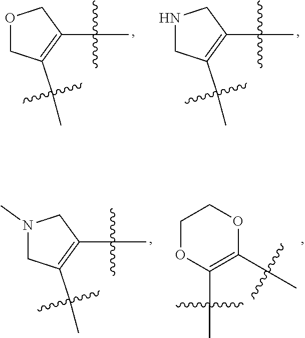

phenyl ring, N-methylpyrrole ring, furan ring or thiophene ring;
R² is selected from the group consisting of H and C₁₋₆ alkyl;
R³, R⁴, R⁷, R⁷ and R⁸, at each occurrence, are each independently selected from the group consisting of H, halogen, —NH₂, —OH, C₁₋₆ alkyl and —OR⁵;
R⁹ and R¹⁰, at each occurrence, are each independently selected from the group consisting of H, halogen, C₁₋₆ alkyl, C₂₋₆ alkenyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵ and -C₁₋₆ alkylene-O(P═O)(OH)₂;
the above alkyl, alkenyl, cyclic hydrocarbyl, heterocyclyl, aryl, heteroaryl, heteroaromatic ring and aralkyl, at each occurrence, are each optionally substituted with one or more substituents independently selected from the group consisting of halogen, C₁₋₆ alkyl and —OR⁵;
R⁵ and R⁶, at each occurrence, are each independently selected from the group consisting of H, C₁₋₆ alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3; and
n is an integer of 0, 1 or 2.

In preferred embodiments, R⁵ and R⁶, at each occurrence, are each independently selected from the group consisting of H, methyl and ethyl.

In preferred embodiments, R³, R⁴, R⁷, R^{7′} and R⁸, at each occurrence, are each independently selected from the group consisting of H, F, Cl, Br, —NH₂, —OH, methyl, trifluoromethyl, —CH₂—Ph, methoxy, ethoxy and —CH₂OCH₃.

In preferred embodiments, R⁹ and R¹⁰, at each occurrence, are each independently selected from the group consisting of H, F, Cl, Br, methyl, ethyl, n-propyl, isopropyl, vinyl, cyclopropyl, cyclobutyl, cyclopentyl, oxetanyl, monofluoromethyl, difluoromethyl, trifluoromethyl, acetyl, —OCH₂CHF₂, CH₂OH, —CH₂OCH₃, —CH₂CH₂OCH₃, —CH₂—O(P═O)(OH)₂,

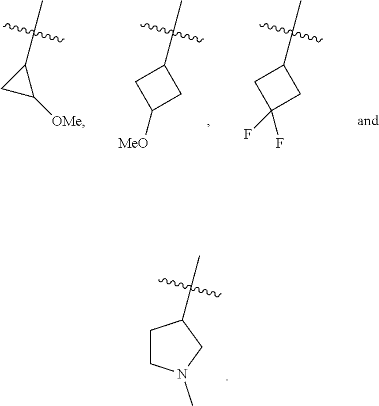

In one embodiment, the present disclosure provides a method for treating the viral infections, diseases of conditions disclosed herein using a compound (6-(4-((4-(1H-pyrazol-4-yl)phenyl)amino)pyrimidin-2-yl)-1-methyl-1H-indol-2-yl)(3,3-difluoroazetidin-1-yl)methanone having the chemical formula XII

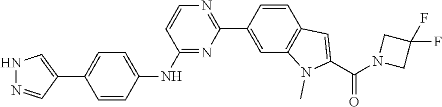 [SMILES: Cn1c(C(=O)N2CC(F)(F)C2)cc2ccc(-c3nccc(Nc4ccc(-c5cn[nH]c5)cc4)n3)cc21]

or a pharmaceutically acceptable salt, ester, stereoisomer, polymorph, solvate, N-oxide, or isotopically labeled compound thereof.

Compounds of the Formula I to XII, and particularly (6-(4-((4-(1H-pyrazol-4-yl)phenyl)amino)pyrimidin-2-yl)-1-methyl-1H-indol-2-yl)(3,3-difluoroazetidin-1-yl)methanone, may be prepared according to the methods disclosed in U.S. 2019/0276440, the contents of which is incorporated herein in its entirety.

As used herein, the term “alkylene” refers to a saturated divalent hydrocarbyl, preferably refers to a saturated divalent hydrocarbyl having 1, 2, 3, 4, 5 or 6 carbon atoms, e.g., methylene (—CH₂—), ethylene (—CH₂CH₂—), propylene or butylene.

As used herein, the term “alkyl” is defined as a linear or branched saturated aliphatic hydrocarbon. In some embodiments, alkyl has 1-12, e.g., 1-6, carbon atoms. For example, as used herein, the term “C₁₋₆ alkyl” refers to a linear or branched group having 1-6 carbon atoms (such as methyl, ethyl, n-propyl, isopropyl, n-butyl, isobutyl, sec-butyl, tent-butyl, n-pentyl, isopentyl, neopentyl, or n-hexyl), which is optionally substituted with one or more (e.g., 1 to 3) suitable substituents such as halogen (in which case the group may be referred to as “haloalkyl”) (e.g., CH₂F, CHF₂, CF₃, CCl₃, C₂F₅, C₂Cl₅, CH₂CF₃, CH₂Cl or CH₂CH₂CF₃, etc.). The term “C₁₋₄ alkyl” refers to a linear or branched aliphatic hydrocarbon chain having 1-4 carbon atoms (i.e., methyl, ethyl, n-propyl, isopropyl, n-butyl, isobutyl, sec-butyl or tent-butyl).

As used herein, the term “alkenyl” refers to a linear or branched monovalent hydrocarbyl having a double bond and 2-6 carbon atoms (“C₂₋₆ alkenyl”). The alkenyl is e.g., vinyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl and 4-methyl-3-pentenyl. When the compound of the present disclosure contains an alkenylene group, the compound may exist as the pure E (entgegen) form, the pure Z (zusammen) form, or any mixture thereof.

As used herein, the term “alkynyl” refers to a monovalent hydrocarbyl containing one or more triple bond, and preferably having 2, 3, 4, 5 or 6 carbon atoms, e.g., ethynyl or propynyl.

As used herein, the term “cycloalkyl” refers to a saturated monocyclic or polycyclic (e.g., bicyclic) hydrocarbon ring (e.g., monocyclic, such as cyclopropyl, cyclobutyl, cyclopentyl, cyclohexyl, cycloheptyl, cyclooctyl, or cyclononyl, or bicyclic, including spiro, fused or bridged cyclic system (such as bicyclo [1.1.1] pentyl, bicyclo [2.2.1] heptyl, bicyclo [3.2.1] octyl or bicyclo [5.2.0] nonyl, or decahydronaphthalene etc.)), which is optionally substituted with one or more (e.g., 1 to 3) suitable substituents. The cycloalkyl has 3 to 15 carbon atoms. For example, the term “C₃₋₆ cycloalkyl” refers to a saturated monocyclic or polycyclic (e.g., bicyclic) hydrocarbon ring having 3 to 6 ring forming carbon atoms (e.g., cyclopropyl, cyclobutyl, cyclopentyl, or cyclohexyl), which is optionally substituted with one or more (e.g., 1 to 3) suitable substituents, e.g., methyl substituted cyclopropyl.

As used herein, the terms “cyclic hydrocarbylene”, “cyclic hydrocarbyl” and “hydrocarbon ring” refer to a saturated (i.e., “cycloalkylene” and “cycloalkyl”) or unsaturated (i.e., having one or more double and/or triple bonds in the ring) monocyclic or polycyclic hydrocarbon ring having e.g., 3-10 (suitably having 3-8, and more suitably having 3-6) ring carbon atoms, including but not limited to cyclopropyl(ene) (ring), cyclobutyl(ene) (ring), cyclopentyl(ene) (ring), cyclohexyl(ene) (ring), cycloheptyl(ene) (ring), cyclooctyl(ene) (ring), cyclononyl(ene) (ring), cyclohexenyl(ene) (ring), and the like.

As used herein, the terms “heterocyclyl”, “heterocyclylene” and “heterocycle” refer to a saturated (i.e., heterocycloalkyl) or partially unsaturated (i.e., having one or more double and/or triple bonds in the ring) cyclic group having e.g. 3-10 (suitably having 3-8, and more suitably having 3-6) ring atoms, wherein at least one ring atom is a heteroatom selected from the group consisting of N, O and S, and the remaining ring atoms are C. For example, “3- to 10-membered heterocyclyl(ene)” of “3- to 10-membered heterocycle” refers to saturated or partially unsaturated heterocyclyl(ene) or heterocycle having 2-9 (e.g., 2, 3, 4, 5, 6, 7, 8 or 9) ring carbon atoms and one or more (e.g., 1, 2, 3, or 4) heteroatoms independently selected from the group consisting of N, O and S. Examples of heterocyclylene, heterocyclyl and heterocycle include, but are not limited to oxiranyl(ene), aziridinyl(ene), azetidinyl(ene), oxetanyl(ene), tetrahydrofuranyl(ene), dioxolinyl(ene), pyrrolidinyl(ene), pyrrolidonyl(ene), imidazolidinyl(ene), pyrazolidinyl(ene), pyrrolinyl(ene), tetrahydropyranyl(ene), piperidinyl(ene), morpholinyl(ene), dithianyl(ene), thiomorpholinyl(ene), piperazinyl(ene) or trithianyl(ene). Said group also encompasses a bicyclic system, including a spiro, fused, or bridged system (e.g., 8-azaspiro[4.5]decane, 3,9-diazaspiro[5.5]undecane, 2-azabicyclo[2.2.2]octane, etc.). Heterocyclylene, heterocyclyl and heterocycle may optionally be substituted with one or more (e.g. 1, 2, 3 or 4) suitable substituents.

As used herein, the terms “aryl(ene)” and “aromatic ring” refer to an all-carbon monocyclic or fused-ring polycyclic aromatic group having a conjugated π electron system. For example, as used herein, the terms “C₆₋₁₀ aryl(ene)” and “C₆₋₁₀ aromatic ring” refer to an aromatic group containing 6 to 10 carbon atoms, such as phenyl(ene) (benzene ring) or naphthyl(ene) (naphthalene ring). Aryl(ene) or aromatic ring is optionally substituted with one or more (such as 1 to 3) suitable substituents (e.g., halogen, —OH, —CN, —NO₂, and C₁₋₆ alkyl, etc.).

As used herein, the terms “heteroaryl(ene)” and “heteroaromatic ring” refer to a monocyclic, bicyclic or tricyclic aromatic ring system having 5, 6, 8, 9, 10, 11, 12, 13 or 14 ring atoms, particularly 1 or 2 or 3 or 4 or 5 or 6 or 9 or 10 carbon atoms, and containing at least one heteroatom (such as O, N, or S), which can be same to different. Moreover, in each case, it can be benzo-fused. In particular, “heteroaryl(ene)” or “heteroaromatic ring” is selected from the group consisting of thienyl(ene), furyl(ene), pyrrolyl(ene), oxazolyl(ene), thiazolyl(ene), imidazolyl(ene), pyrazolyl(ene), isoxazolyl(ene), isothiazolyl(ene), oxadiazolyl(ene), triazolyl(ene), thiadiazolyl(ene) etc., and benzo derivatives thereof; or pyridinyl(ene), pyridazinyl(ene), pyrimidinyl(ene), pyrazinyl(ene), triazinyl(ene), etc., and benzo derivatives thereof.

As used herein, the term “aralkyl” preferably means aryl or heteroaryl substituted alkyl, wherein aryl, heteroaryl and alkyl are as defined herein. Normally, the aryl group may have 6-14 carbon atoms, the heteroaryl group may have 5-14 ring atoms, and the alkyl group may have 1-6 carbon atoms. Exemplary aralkyl group includes, but is not limited to, benzyl, phenylethyl, phenylpropyl, phenylbutyl.

As used herein, the term “halo” or “halogen” are defined to include F, Cl, Br, or I.

As used herein, the term “nitrogen containing heterocycle” refers to a saturated or unsaturated monocyclic or bicyclic group having 2, 3, 4, 5, 6, 7, 8, 9, 10, 11, 12 or 13 carbon atoms and at least one nitrogen atom in the ring, which may further optionally comprise one or more (e.g., one, two, three or four) ring members selected from the group consisting of N, O, C═O, S, S═O and S(═O)₂. The nitrogen containing heterocycle is attached to the rest of the molecule through the nitrogen atom and any other ring atom in said nitrogen containing heterocycle. The nitrogen containing heterocycle is optionally benzo-fused and is preferably attached to the rest of the molecule through the nitrogen atom in said nitrogen containing heterocycle and any carbon atom in the fused benzene ring.

The term “substituted” means that one or more (e.g., one, two, three, or four) hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent.

If substituents are described as being “independently selected” from a group, each substituent is selected independent of the other(s). Each substituent therefore may be identical to or different from the other substituent(s).

As used herein, the term “one or more” means one or more than one (e.g., 2, 3, 4, 5 or 10) as reasonable.

As used herein, unless specified, the point of attachment of a substituent can be from any suitable position of the substituent.

When a bond to a substituent is shown to cross a bond connecting two atoms in a ring, then such substituent may be bonded to any of the ring-forming atoms in that ring that are substitutable.

The compounds for use in the methods provided herein include pharmaceutically acceptable isotopically labeled compounds, which are identical to those of Formulas I to XII, except that one or more atoms are replaced by an atom having the same atomic number, but an atomic mass or mass number different from the atomic mass or mass number which predominates in nature. Examples of isotopes suitable for inclusion in the compounds include, but are not limited to, isotopes of hydrogen, such as ²H, ³H; carbon, such as ¹¹C, ¹³C, and ¹⁴C; chlorine, such as ³⁶ Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S. Certain isotopically labeled compounds of the present disclosure, for example those incorporating a radioactive isotope, are useful in drug and/or substrate tissue distribution studies (e.g., assays). The radioactive isotopes tritium, i.e., ³H, and carbon-14, i.e., ¹⁴C are particularly useful for this purpose in view of their ease of incorporation and ready means of detection. Substitution with positron-emitting isotopes, such as ¹¹C, ¹⁸F, ¹⁵O and ¹³N, can be useful in positron emission tomography (PET) studies for examining substrate receptor occupancy. Isotopically labeled compounds of the present disclosure can generally be prepared by processes analogous to those described in the accompanying Schemes and/or in the Examples and Preparations, by using an appropriate isotopically labeled reagent in place of the non-labeled reagent previously employed. Pharmaceutically acceptable solvates in accordance with the disclosure include those wherein the solvent of crystallization may be isotopically substituted, e.g., D₂O, acetone-d₆, or DMSO-d₆.

The term “stereoisomer” refers to isomers with at least one asymmetric center. A compound having one or more (e.g., one, two, three or four) asymmetric centers can give rise to a racemic mixture, single enantiomer, diastereomer mixture and individual diastereomer. Certain individual molecules may exist as geometric isomers (cis/trans). Similarly, the compounds provided herein may exist as a mixture of two or more structurally different forms in rapid equilibrium (generally referred to as tautomer). Typical examples of a tautomer include a keto-enol tautomer, phenol-keto tautomer, nitroso-oxime tautomer, imine-enamine tautomer and the like. It is to be understood that the use of all such isomers and mixtures thereof in any proportion (such as 60%, 65%, 70%, 75%, 80%, 85%, 90%, 95%, 96%, 97%, 98%, and 99%) are encompassed within the scope of the present disclosure.

The present disclosure includes the use of crystalline forms or polymorphs of the compounds disclosed herein, either as a single polymorph, or as a mixture of more than one polymorphs, in any ratio.

It also should be understood that certain compounds as provided herein can be used for the treatment in a free form, or where appropriate, in a form of a pharmaceutically acceptable derivative. In the present disclosure, the pharmaceutically acceptable derivative includes, but is not limited to a pharmaceutically acceptable salt, ester, solvate, N-oxide, metabolite, or prodrug, which can directly or indirectly provide the compound of the present disclosure or a metabolite or residue thereof after being administered to a patient in need thereof. Therefore, the compounds provided herein encompass various derivative forms of the compound as mentioned above.

A pharmaceutically acceptable salt of the compounds disclosed herein includes an acid addition salt or a base addition salt. A suitable acid addition salt is formed from an acid which forms a pharmaceutically acceptable salt. Specific, non-limiting examples include acetate, adipate, aspartate, benzoate, besylate, bicarbonate/carbonate, bisulfate/sulfate, borate, camphorsulfonate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. A suitable base addition salt is formed from a base which forms a pharmaceutically acceptable salt. Specific, non-limiting examples include aluminum, arginine, benzathine, calcium, choline, diethylamine, diolamine, glycine, lysine, magnesium, meglumine, olamine, potassium, sodium, tromethamine and zinc salts.

For a review on suitable pharmaceutically acceptable salts, see “Handbook of Pharmaceutical Salts: Properties, Selection, and Use” by Stahl and Wermuth (Wiley-VCH, 2002).

As used herein, the term “ester” refers to those derived from the compounds of the various formulae provided herein, which include physiologically-hydrolyzable esters (which may be hydrolyzed under physiological conditions to release the compounds of the present disclosure in the form of free acids or alcohols).

The compounds for use in the methods of the present disclosure can exist as a solvate (preferably a hydrate), wherein the compound contains a polar solvent, in particular water, methanol or ethanol for example, as a structural element of the crystal lattice of the compound. The amount of the polar solvent, in particular water, may exist in a stoichiometric or non-stoichiometric ratio.

As can be appreciated by a person skilled in the art, not all nitrogen containing heterocycles can form N-oxides because the nitrogen requires an available electron lone-pair for oxidation to the oxide; a person skilled in the art will recognize those nitrogen containing heterocycles which can form N-oxides. A person skilled in the art will also recognize that tertiary amines can form N-oxides. Synthetic methods for the preparation of N-oxides of heterocycles and tertiary amines are well known to a person skilled in the art, and they include the oxidation of heterocycles and tertiary amines with peroxy acids such as peracetic acid and m-chloroperbenzoic acid (MCPBA), hydrogen peroxide, alkyl hydroperoxides such as tent-butyl hydroperoxide, sodium perborate, and dioxiranes such as dimethyldioxirane. These methods for the preparation of N-oxides have been extensively described and reviewed in literatures, see e.g., T. L. Gilchrist, Comprehensive Organic Synthesis, vol. 7, pp 748-750; A. R. Katritzky and A. J. Boulton, Eds., Academic Press; and G W. H. Cheeseman and E. S. G Werstiuk, Advances in Heterocyclic Chemistry, vol. 22, pp 390-392, A. R. Katritzky and A. J. Boulton, Eds., Academic Press.

The compounds described herein may be administered in the form of a prodrug, in which certain derivatives of the compound that may have little or no pharmacological activity itself, can, when administered into or onto the body, be converted into a compound having the desired activity, for example, by hydrolytic cleavage. In general, such prodrug will be a functional derivative of the compound which is readily converted in vivo into the compound with desired therapeutic activity. Further information on the use of the prodrug may be found in “Pro-drugs as Novel Delivery Systems,” Vol. 14, ACS Symposium Series (T. Higuchi and V. Stella). The prodrug can, for example, be produced by replacing appropriate functionalities present in the compound of the present disclosure with moieties known to those skilled in the art as “pro-moieties” as described, for example, in “Design of Prodrugs” by H. Bundgaard (Elsevier, 1985).

In some embodiments, compounds of the Formulas I to XII are selective inhibitors of Rho-associated coiled-coil kinase 2 (ROCK2) in human cells. Compounds of the Formulas I to XII, for example as a pharmaceutical composition comprising the compound, are used to treat (i.e., cure or reduce the severity of, etc.) viral infections, particularly coronavirus infections such as SARS-CoV-1, SARS-CoV-2, and MERS-CoV, and to treat or prevent the sequelae resulting from the viral infection, including the coronavirus infection such as SARS-CoV-1, SARS-CoV-2, and MERS-CoV. In some embodiments, the viral infection is a SARS-CoV-1 infection. In some embodiments, the viral infection is a SARS-CoV-2 infection. In some embodiments, the viral infection is a MERS-CoV infection.

Methods of determining kinase inhibition are disclosed herein. For example, kinase activity of an enzyme and the inhibitory capacity of a test compound can be determined by measuring enzyme-specific phosphorylation of a substrate. Commercial assays and kits can be employed. For example, kinase inhibition can be determined using an IMAP® assay (Molecular Devices). This assay method involves the use of a fluorescently-tagged peptide substrate. Phosphorylation of the tagged peptide by a kinase of interest promotes binding of the peptide to a trivalent metal-based nanoparticle via the specific, high affinity interaction between the phospho-group and the trivalent metal. Proximity to the nanoparticle results in increased fluorescence polarization. Inhibition of the kinase by a kinase inhibitor prevents phosphorylation of the substrate and thereby limits binding of the fluorescently-tagged substrate to the nanoparticle. Such an assay can be compatible with a microwell assay format, allowing simultaneous determination of the IC₅₀ of multiple compounds.

### Pharmaceutical Compositions

In one aspect, the present disclosure provides pharmaceutically acceptable compositions for use in the treatment of viral diseases which comprise a therapeutically-effective amount of one or more of the compounds of Formula I to XII, formulated together with one or more pharmaceutically acceptable carriers. As described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginal or intrarectal administration, for example, as a suppository, pessary, cream or foam; (5) sublingual administration; (6) ocular administration; (7) transdermal administration; or (8) nasal administration.

The phrase “pharmaceutically acceptable” is employed herein to refer to those compounds, materials, compositions, and/or dosage forms which are, within the scope of sound medical judgment, suitable for use in contact with the tissues of human beings and animals with toxicity, irritation, allergic response, or other problems or complications, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), solvent, or solvent encapsulating material, involved in carrying or transporting the subject compound from one organ, or portion of the body, to another organ, or portion of the body. Each carrier should be compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The compounds of this disclosure may be formulated with conventional carriers and excipients, which can be selected in accord with ordinary practice. Tablets can contain excipients, glidants, fillers, binders and the like. Aqueous formulations are prepared in sterile form, and when intended for delivery by other than oral administration generally can be isotonic. All formulations can optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like.

While it is possible for the ROCK2 inhibitors disclosed herein (herein referred to as the “active ingredients”) to be administered alone, it may be preferable to present them as pharmaceutical formulations. The formulations, both for veterinary and for human use, of the disclosure comprise at least one active ingredient, as provided above, together with one or more acceptable carriers therefor and optionally other therapeutic ingredients, particularly those additional therapeutic ingredients as discussed herein.

The formulations include those suitable for the administration routes provided herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the present disclosure suitable for oral administration may be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues e.g. mouth and skin, the formulations are preferably applied as a topical solution, ointment or cream containing the active ingredient(s). The active ingredient may be present in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this disclosure may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulsifying agents and emulsion stabilizers suitable for use in the formulation of the disclosure include Tween® 60, Span® 80, cetostearyl alcohol, benzyl alcohol, myristyl alcohol, glyceryl mono-stearate and sodium lauryl sulfate. Further emulsifying agents and emulsion stabilizers suitable for use in the formulation of the disclosure include Tween® 80.

The choice of suitable oils or fats for the formulation is based on achieving the desired properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present disclosure comprise a combination according to the disclosure together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example starch, mannitol, calcium phosphate or kaolin, or as soft gelatin capsules wherein the active ingredient is mixed with water or an oil medium, such as peanut oil, liquid paraffin or olive oil.

Aqueous suspensions of the disclosure contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin and Captisol (=Sulfobutyl ether beta-cyclodextrin; SEB-beta-CD).

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the disclosure may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form can vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient may be present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds.

Formulations suitable for vaginal administration may be presented as suppositories, pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The disclosure further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the disclosure are used to provide controlled release pharmaceutical formulations containing as active ingredient one or more compounds of the disclosure (“controlled release formulations”) in which the release of the active ingredient are controlled and regulated to allow less frequency dosing or to improve the pharmacokinetic or toxicity profile of a given active ingredient.

### Combinations With Other Active Agents

The ROCK2 inhibitors disclosed herein may be used in combination with at least one additional therapeutic agent. The at least one additional therapeutic agent may be, for example, an antiviral agent, a corticosteroid, an anti-inflammatory signal transduction modulator, a β2-adrenoreceptor agonist bronchodilator, an anticholinergic, a mucolytic agent, hypertonic saline, or a mixture thereof.

The compound having the Formula I to XII may be administered in combination with one or more additional antiviral therapies and/or antiviral agents. The at least one other antiviral agent may be a nucleoside or nucleotide analog, or a pharmaceutically acceptable salt or prodrugs thereof. The antiviral agent may be selected from remdesivir, ribavirin, favipiravir, T-705 monophosphate, T-705 diphosphate, T-705 triphosphate, ST-193, idoxuridine, edoxudine, trifluridine, vidarabine, brivudine, acyclovir, ganciclovir, valaciclovir, cidofovir, valganciclovir, penciclovir, famciclovir, zidovudine, didanosine, zalcitabine, stavudine, abacavir, lamivudine, emtricitabine, tenofovir disoproxil fumarate, tenofovir alafenamide fumarate, adefovir, entecavir, telbivudine, sofosbuvir, and combinations or mixtures thereof.

It is also possible to combine any compound of the disclosure with one or more additional therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the disclosure with one or more other active therapeutic agents generally refers to simultaneous or sequential administration of a compound of the disclosure and one or more other active therapeutic agents, such that therapeutically effective amounts of the compound of the disclosure and one or more other active therapeutic agents are both present in the body of the patient.

Co-administration includes administration of unit dosages of the compounds of the disclosure before or after administration of unit dosages of one or more additional therapeutic agents, for example, administration of the compounds of the disclosure within seconds, minutes, or hours of the administration of one or more additional therapeutic agents. For example, a unit dose of a compound of the disclosure can be administered first, followed within seconds or minutes by administration of a unit dose of one or more additional therapeutic agents. Alternatively, a unit dose of one or more additional agents can be administered first, followed by administration of a unit dose of a compound of the disclosure within seconds or minutes. In some cases, it may be desirable to administer a unit dose of a compound of the disclosure first, followed, after a period of hours (e.g., 1-12 hours), by administration of a unit dose of one or more additional therapeutic agents. In other cases, it may be desirable to administer a unit dose of one or more additional therapeutic agents first, followed, after a period of hours (e.g., 1-12 hours), by administration of a unit dose of a compound of the disclosure.

The combination therapy may provide “synergy” and “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

### Methods of Treating Viral Infections

As used herein, “patient” and “subject” are used interchangeably.

In one aspect, the disclosure provides methods for treating a viral infection, particularly coronavirus infections. In one aspect, the disclosure provides methods for the treatment and prevention of sequelae resulting from the viral infection, including sequelae resulting from coronavirus infection. In one aspect, the disclosure provides methods of preventing a viral infection in a subject at risk for the viral infection, including at risk for a coronavirus infection.

In embodiments, the viral infection is caused by a virus selected from the group consisting of SARS-CoV-1, SARS-CoV-2, MERS-CoV, Yellow Fever, Eastern Equine Encephalitis virus, Human Immunodeficiency virus (HIV), African Swine Fever viruses, Arbovirus, Adenoviridae, Arenaviridae, Arterivirus, Astroviridae, Baculoviridae, Bimaviridae, Bimaviridae, Bunyaviridae, Caliciviridae, Caulimoviridae, Circoviridae, Coronaviridae, Cystoviridae, EBV, Deltaviridae, Filviridae, Filoviridae, Flaviviridae, Iridoviridae, Mononegavirus, Myoviridae, Papiloma virus, Papovaviridae, Paramyxoviridae, Prions, Parvoviridae, Phycodnaviridae, Poxviridae, Potyviridae, Reoviridae, Retroviridae, Rhabdoviridae, Tectiviridae, Togaviridae, pox, papilloma, influenza, sendai virus (SeV), sindbis virus (SINV), vaccinia viruses, West Nile, Hanta, viruses which cause the common cold, and any combination thereof. In embodiments, the viral infection is caused by a coronavirus, such as SARS-CoV-1, SARS-CoV-2 and MERS-CoV. In embodiments, the viral infection is caused by SARS-CoV-1. In embodiments, the viral infection is caused by SARS-CoV-2. In embodiments, the viral infection is caused by one or more SARS-CoV-2 variants selected from the group consisting of Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), 1.617.3, Mu (B.1.621, B.1.621.1), Zeta (P.2), Delta (B.1.617.2 and AY lineages), and Omicron (B.1.1.529 and BA lineages). In embodiments, the viral infection is caused by SARS-CoV-2 variant Delta and/or SARS-CoV-2 variant Omicron.

In embodiments, the viral infection is caused by MERS-CoV.

In embodiments, the disclosure provides a method for treating or preventing one or more sequelae of COVID-19, comprising administering to a subject in need thereof a therapeutically effective amount of a compound of Formulas I to XII. In embodiments, the disclosure provides a method for treating or preventing one or more sequelae of COVID-19, comprising administering to a subject in need thereof a therapeutically effective amount of a compound of Formula XII.

Sequelae of coronavirus infection, and in particular SARS-CoV-1, SARS-CoV-2 and MERS-CoV may result from one or more phenomenon including organ damage from the acute infection phase, manifestations of a persistent hyperinflammatory state, ongoing viral activity associated with a host viral reservoir, or an inadequate antibody response. Sequelae of COVID-19 include fatigue, dyspnea (difficulty breathing), cough, arthralgia (joint pain), and chest pain. Additional sequelae include cognitive impairment, depression, myalgia, headache, fever, and palpitations. Sequelae of COVID-19 may be cardiovascular (e.g., myocardial inflammation, ventricular dysfunction, stroke), respiratory (e.g., pulmonary function abnormalities, fibrosis), renal (e.g., acute kidney injury), dermatologic (e.g., rash, alopecia), neurological (e.g., olfactory and gustatory dysfunction, sleep dysregulation, altered cognition, memory impairment), and/or psychiatric (e.g., depression, anxiety, changes in mood).

Without being bound by theory, a ROCK2 inhibitor, such as, for example, compounds of the Formulas I to XII, may block: (1) pathways used by the virus to enter cells; (2) the cellular cytoskeleton used by the virus for migration and spread; (3) pathways used by the virus to upregulate the energy metabolism of a cell; and (4) pathways used by the virus to spread to other cells.

The ROCK2 inhibitors provided herein may interfere with the interaction of the virus with the cytoskeleton of the host cell, and thereby inhibit the entry, replication, and/or spread of the virus. The cytoskeleton is an intricate network in eukaryotic cells, which comprises three major types of cytoskeletal polymers including actin filaments, microtubules, and intermediate filaments, allowing cells to perform multiple functions in a united way, such as connecting to the external environment, coordinating forces to move and change shapes, transporting vesicles through the cytoplasm, and spatially organizing the contents. Most viruses hijack one or more aspects of the cytoskeleton network to facilitate their own infection. The viral interaction with host cell actin filaments, microtubules, and intermediate filaments play the important roles in the life cycle of viruses, and particularly of coronaviruses.

The ROCK2 inhibitors provided herein may interfere with the entry of a virus into its target cell. After binding to the target cell, viruses may migrate to favorable sites for entry. As the virus reaches the entry site, actin filaments have been observed to retract and concentrate around the plasma membrane. Pharmacological stabilization of actin cortex, may interfere with the viral penetration of the host cells. Viruses also take advantage of cytoskeleton-regulating signaling pathways as part of their infection processes.

Coronavirus may utilize the three cytoskeleton networks to complete viral transport process. Transport from/to the cell periphery for short-range route is mediated by actin and its motor proteins like myosin, while long-range transport is mediated by microtubules and the motor proteins dynein and kinesin. After entering host cell, coronavirus-containing vesicles run along microtubules to move from the plasma membrane toward replication sites.

SARS-CoV-2-infected cells can fuse with neighboring cells to form actin-regulated syncytia. During the infection stage, SARS-CoV-2 surfs along filopodia on the host membrane to the cell entry sites and uses intermediate filament proteins to assist and utilize angiotensin converting enzyme-2 (ACE2) receptors to enter target cells. In a mouse SARS model, the SARS-CoV spike protein bound ACE2 receptors and reduced ACE2 expression leading to severe lung injury. By binding to ACE2 receptors to gain cell entry, SARS-CoV-2 effectively blocks ACE2 activity and the conversion of AT-II to Angiotensin, leading to vasoconstriction, increased vascular permeability, adverse myocardial remodeling, and acute lung injury. The downregulation of ACE2 and interruption of the normal feedback loop can also lead to a life-threatening cytokine storm as commonly observed in moderate to severe cases of COVID-19. An excessive inflammatory response to SARS-CoV-2 represents the main cause of disease severity and death in COVID-19 patients.

When ROCK2 is inhibited, ACE2 activity is restored, AT-II conversion continues, and inflammatory cytokines (i.e., IL-1β, IL-6, and TNF-alpha), TGF-β1, and pro-fibrotic markers (i.e., COL1A1, COL3A1, alpha-SMA, fibronectin, CTGF, and PDGF-B) are downregulated. ROCK inhibition also can play a role in preventing viral spreading between host cells, by inhibiting Rho GTPase mediated cytoskeletal reorganization including movement, migration, survival, cell death, and the formation of and viral syncytia.

Additionally, the neurodevelopmental disorders and respiratory tract damage caused by coronaviruses are, at least in part, microtubule-dependent. Structural damage to the respiratory epithelium and abnormal ciliary function are typical pathologic symptoms of coronavirus infection. Cilia is a composite structure present on the cell surface comprised of microtubules. Coronaviruses that cause severe respiratory damage may do so through cilia loss in the upper respiratory tract and lung. Further, disruption of microtubules may be related to neurodegenerative diseases.

Based on its multifaceted modes of action, ROCK2 inhibitors of Formulas I to XII inhibit or prevent viral cell entry and inhibit viral spread, for example by inhibiting the syncytia. Secondarily, the ROCK2 inhibition also mitigate the overactive immune response known as “cytokine storm”, as well as the fibrotic changes in the vascular, cardiac, and lung tissues which have resulted in long-term sequalae for some COVID-19 patients.

In some embodiments, the ROCK2 inhibitor: (a) inhibits viral entry of the viral infection, and/or (b) modulates the viral entry of the viral infection.

In some embodiments, the ROCK2 inhibitor: (a) inhibits viral spread of the viral infection, and/or (b) modulates the viral spread of the viral infection.

In some embodiments, the ROCK2 inhibitor targets a cellular target of the subject. Because the ROCK2 inhibitor interacts with a cellular target of the host, rather than with a viral target, the development of drug resistance to the effects of the ROCK2 inhibitor in the virus may be slowed, or substantially non-existent, when compared with other antiviral agents or compositions. In some embodiments, the use of the ROCK2 inhibitor for the treatment of viral disease is characterized by the absence of drug resistance.

In some embodiments, the ROCK2 inhibitor inhibits stress fiber formation, and/or modulates the stress fiber formation.

In some embodiments, the ROCK2 inhibitor inhibits actin filament dynamics, and/or modulates the actin filament dynamics.

In some embodiments, the ROCK2 inhibitor inhibits the mammalian target of rapamycin (mTOR) metabolic pathway, and/or modulates the mTOR metabolic pathway.

In some embodiments, the ROCK2 inhibitor inhibits the AKT (serine/threonine protein kinase B) metabolic pathway, and/or modulates the AKT metabolic pathway.

In some embodiments, the ROCK2 inhibitor inhibits fusogenic pathway, and/or modulates the fusogenic pathway.

In some embodiments, the human subject is an adult patient. In embodiments, the human subject has, or is at risk of having, fibrosis or scarring on lungs. In some embodiments, the human subject is a pediatric patient. In embodiments, the human subject has, or is at risk of developing, Kawasaki disease or fibrosis or scarring on the lungs.

In embodiments, the ROCK2 inhibitor at least partially reverses and/or inhibits the level of fibrosis, at least partially inhibits the over-deposition of extracellular matrix in the lungs, or improves blood supply to the lungs. In some embodiments, the administration of the ROCK2 inhibitor results in at least one of the following: (a) at least 5% reduction of lung edema; (b) at least 5% reduction of lung pathology severity scores associated with lung fibrosis; (c) at least 5% reduction of expression of pro-inflammatory proteins, or (d) at least 5% reduction of expression of fibrogenic proteins.

In some embodiments, the human subject experiences a decline of forced vital capacity of: (a) less than 10%; (b) less than 9%; (c) less than 8%; (d) less than 7%; (e) less than 6%; (f) less than 5%; (g) less than 4%; (h) less than 3%; (i) less than 2%; or (j) less than 1%; following administration of the ROCK2 antagonist to the human subject for at least 2 weeks. In some embodiments, the human subject experiences no decline of forced vital capacity following administration of the ROCK2 antagonist to the human subject for at least 2 weeks. In some embodiments, the human subject experiences an increase of forced vital capacity of: (a) at least 0.5%; (b) at least 1%; (c) at least 1.5%; (d) at least 2%; (e) at least 2.5%; (f) at least 3%; (g) at least 3.5%; (h) at least 4%; (i) at least 4.5%; or (j) at least 5%; following administration of the ROCK2 antagonist to the human subject for at least 2 weeks.

In some embodiments, the human subject experiences a decrease of occurrence of coronary artery lesions at one month of illness of: (a) at least 5%; (b) at least 10%; (c) at least 15%, (d) at least 20%; (e) at least 25%; (f) at least 30%; (g) at least 35%; or (h) at least 40%; following administration of the ROCK2 antagonist to the human subject for at least 2 weeks. In some embodiments, the ROCK2 antagonist at least partially reverses or reduces vasculitis syndrome, or at least partially reverses or reduces rash, redness to eyes, lips, or tongue, swelling of hands or feet, redness to hands or feet, or neck swelling. In some embodiments, the ROCK2 antagonist rebalances an immune response in the human subject.

In one aspect, the present disclosure provides a method of treating a patient suffering from a viral disease comprising administering to a patient in need of such treatment a therapeutically effective amount of a compound of Formula I to XII. The phrase “therapeutically-effective amount” as used herein means that amount of a compound, material, or composition comprising a compound of the present disclosure which is effective for producing some desired therapeutic effect in at least a sub-population of cells in the subject.

One or more compounds of the disclosure are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It can be appreciated that the preferred route may vary with for example the condition of the recipient. An advantage of the compounds of this disclosure is that they are orally bioavailable and can be dosed orally.

Effective dose of the compounds of the disclosure depends at least on the nature of the condition being treated, toxicity, whether the compound is being used prophylactically or against an active viral infection, the method of delivery, and the pharmaceutical formulation, and can be determined by the clinician using conventional dose escalation studies. In embodiments, doses of the compounds of the disclosure range from about 0.1 to about 50 mg/kg body weight per day. The daily dose for adult human may range from 1 mg to 1000 mg, for example between about 5 mg and about 800 mg or between about 50 mg and 500 mg and may take the form of single or multiple doses per day. In embodiments, the daily dose of a compound of Formulas I-XII to treat the viral infection or treat or prevent one or more sequelae due to the infection is about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg, which may be administered as a single daily dose, or split between two, three or more daily administrations. In embodiments, the dose is a total about 200 mg administered in one daily administration. In embodiments, the dose is a total of about 200 mg administered in two daily administrations. In embodiments, the dose is a total of about 300 mg administered in one daily administration. In embodiments, the dose is a total of about 300 mg administered in two daily administrations. In embodiments, the dose is a total of about 400 mg administered in one daily administration. In embodiments, the dose is a total of about 400 mg administered in two daily administrations.

In embodiments, a compound of the disclosure is first administered to a subject within about 1, about 2, about 3, about 4, about 5, about 6, about 7, about 8, about 9, about 10, about 11, about 12, about 24, about 28, about 72, about 96, or about 120 hours after the subject was exposed to a virus. In embodiments, a compound of the disclosure is first administered to a subject within about 1, about 2, about 3, about 4, about 5, about 6, about 7, about 8, about 9, about 10, about 11, about 12, about 24, about 48, or about 72 hours after the subject has developed symptoms in response to exposure with a virus. In embodiments, a compound of the disclosure is first administered to a subject within about 1, about 2, about 3, about 4, about 5, about 6, about 7, about 8, about 9, about 10, about 12, about 11, about 12, about 13, or about 14 days after the subject has developed symptoms in response to exposure with a virus. In embodiments, a compound of the disclosure is administered prophylactically.

In embodiments, a compound of the disclosure is administered to a subject every day, every other day, every couple of days, every third day, once a week, twice a week, three times a week, once every two weeks, or once a month.

In embodiments, a compound of the disclosure is administered to a subject for about 1, about 2, about 3, about 4, about 5, about 6, or about 7 days. In embodiments, a compound of the disclosure is administered to a subject for about 1, about 2, about 3, about 4, about 5, about 6, or about 7, about 8, about 9, or about 10 weeks. In embodiments, a compound of the disclosure is administered to a subject for about 1 month, about 1.5 months, about 2 months, about 2.5 months, about 3 months, about 4 months, about 5 months, about 6 months or more.

In embodiments, a compound of the disclosure is administered to a subject for about 1, about 2, about 3, about 4, about 5, about 6, or about 7 days after the subject was first exposed to a virus. In embodiments, a compound of the disclosure is administered to a subject for about 1, about 2, about 3, about 4, about 5, about 6, or about 7, about 8, about 9, or about 10 weeks after the subject was first exposed to a virus. In embodiments, a compound of the disclosure is administered to a subject for about 1 month, about 1.5 months, about 2 months, about 2.5 months, about 3 months, about 4 months, about 5 months, about 6 months or more after the subject was first exposed to a virus.

In embodiments, a compound of the disclosure is administered to a subject for about 1, about 2, about 3, about 4, about 5, about 6, or about 7 days after the subject has developed symptoms in response to exposure with a virus. In embodiments, a compound of the disclosure is administered to a subject for about 1, about 2, about 3, about 4, about 5, about 6, or about 7, about 8, about 9, or about 10 weeks after the subject has developed symptoms in response to exposure with a virus. In embodiments, a compound of the disclosure is administered to a subject for about 1 month, about 1.5 months, about 2 months, about 2.5 months, about 3 months, about 4 months, about 5 months, about 6 months or more after the subject has developed symptoms in response to exposure with a virus.

In embodiments, a compound of the disclosure is administered to a subject for about 1, about 2, about 3, about 4, about 5, about 6, or about 7 days after the symptoms that a subject has developed in response to exposure to a virus have subsided. In embodiments, a compound of the disclosure is administered to a subject for about 1, about 2, about 3, about 4, about 5, about 6, or about 7, about 8, about 9, or about 10 weeks after the symptoms that a subject has developed in response to exposure to a virus have subsided. In embodiments, a compound of the disclosure is administered to a subject for about 1 month, about 1.5 months, about 2 months, about 2.5 months, about 3 months, about 4 months, about 5 months, about 6 months or more after the symptoms that a subject has developed in response to exposure to a virus have subsided.

### Methods of Treating Kawasaki Disease/Symptoms Associated With Virus Infection

Among pediatric patients suffering from COVID-19, some display pediatric multisystem inflammatory syndrome (PMIS) or pediatric inflammatory multisystem syndrome temporally associated with SARS-CoV-2 (PIMS-TS), which resembles an inflammatory illness called Kawasaki disease. As used herein, the term “Kawasaki disease” generally refers to an inflammatory disease that causes vasculitis syndrome or inflammation of the blood vessels, sometimes swollen throughout the body, including Kawasaki-like disease such as PMIS or PIMS-TS. Symptoms of Kawasaki disease may include a persistent high fever (over 101° F.) for at least four days in addition to rash, redness to eyes, lips/tongue, swelling and redness to hands/feet and neck swelling, etc. As used herein, the term “Kawasaki disease shock syndrome” or KDSS refers to Kawasaki disease patients who present more than 20% decrease in systolic blood pressure compared to healthy subjects of the same age, or to those patients who show peripheral blood circulation perfusion disorder. KDSS can be due to a high level of circulating inflammatory factors.

Among the pediatric patients of Kawasaki associated with COVID-19, PMIS or PIMS-TS, there may be an increase in inflammatory markers including, for example, neutrophil-predominant leukocytosis, C-reactive protein (CRP), procalcitonin (PCT) and IL-6. The development of Kawasaki or PMIS associated with COVID-19 can be caused by a post-viral immunological reaction, such as, for example, antibody or immune-complex mediated reactions.

As a form of systemic vasculitis, Kawasaki disease may involve small to medium-sized blood vessels. The most severe complication or sequela may be the formation of coronary artery lesions (CAL), such as myocardial infarction, coronary artery fistula, coronary artery dilatation, and coronary artery aneurysm, which may subsequently result in long-term sequelae like stenosis or obstruction and myocardial infarction. Accordingly, the decrease of the rate of coronary artery aneurysms among Kawasaki patients may be an indicator of the effectiveness of the treatment.

The ROCK2 inhibitors of Formula I to XII can reduce pro-inflammatory cytokines, such as, for example, peripheral blood levels of IL-17 and IL-23. In addition, these ROCK2 inhibitors may rebalance the immune response in pediatric patients suffering from Kawasaki disease, PMIS, or PIMS-TS, after infection with COVID-19 or other coronaviruses.

### Methods of Treating Fibrosis Caused by Virus Infection

COVID-19 patients may develop scarring in their lungs either during the acute stage of the disease or after they recover from the illness. The scarring in the lung may be fibrosis or fibrotic scarring. The underlying causes and the clinical course for scarring in the lung in COVID-19 patients may be the same as or different from those with interstitial lung disease and pulmonary fibrosis, such as idiopathic pulmonary fibrosis (IPF) or rheumatoid arthritis.

Fibrosis is the overgrowth, hardening, and/or scarring of various tissues and can be attributed to excess deposition of extracellular matrix components, such as, for example, collagen. Fibrosis may be the result of chronic inflammatory reactions induced by a variety of stimuli, including, for example, persistent infections, autoimmune reactions, allergic responses, chemical insults, radiation, and tissue injury.

COVID-19 infection can lead to a variety of respiratory diseases ranging from atypical pneumonia to acute respiratory distress syndrome (ARDS). Many patients infected by the viruses may share the characteristics of patients suffering from idiopathic pulmonary fibrosis (IPF). For an IPF patient, lung function can inexorably decline, resulting in respiratory failure and death. A possible cause for the lung damage can be a cytokine release syndrome triggered by the viral antigen.

The ROCK2 signaling pathway controls cellular movement and shape. In addition, ROCK2 may regulate cytokine secretion in T cells, such as, for example, promoting pro-inflammatory cytokines such as IL-17 and IL-21, whereas secretion of anti-inflammatory cytokines IL-2 and IL-10 is negatively regulated by ROCK2 under Th17-skewing activation. Also, in disease, but not in steady state conditions, ROCK2 contributes to regulation of IFN-γ secretion in T cells from rheumatoid arthritis patients. Thus, ROCK2 signaling is a key pathway in modulation of T-cell mediated immune responses underscoring the therapeutic potential of targeted inhibition of ROCK2 in autoimmunity. Accordingly, ROCK2 inhibitors can down-regulate IL-21 and IL-17 secretion in human T cells via STAT3-dependent mechanism.

The ROCK2 inhibitors of Formulas I to XII can reduce peripheral blood levels of IL-17 and IL-23, two pro-inflammatory cytokines. In addition, these ROCK2 inhibitors can concurrently up-regulate the immunosuppressive cytokine IL-10 and increase the percentage of Foxp3+ CD4 T cells in blood, which may diminish immuno-inflammatory responses. Thus, the ROCK2 inhibitors having the Formulas I to XII can rebalance the immune response in patients suffering from fibrosis after infection with SARS-CoV-2 or other coronaviruses.

A number of pulmonary function parameters can be used to determine an effective amount of the ROCK2 inhibitor, i.e., an amount to reduce, stabilize or reverse a pathologic rate of decline in one or more pulmonary function parameters in a patient suffering from a viral infection, such as a coronavirus infection; or to monitor patient response to ROCK2 inhibitor therapy. These pulmonary function parameters may include vital capacity (VC), forced expiratory volume (FEV), forced vital capacity (FVC), and FVC %.

As used herein, the term “vital capacity” (VC) refers to the total volume of air that can be moved in and out of the lungs. VC is equal to the combined inspiratory reserve volume, tidal volume, and expiratory reserve volume.

As used herein, the term “forced expiratory volume” (FEV) refers to measuring how much air a subject can exhale during a forced breath. The amount of air exhaled may be measured during the first (FEV1), second (FEV2), and/or third seconds (FEV3) of the forced breath. For example, FEV1/FVC ratio refers to the ratio between forced expiratory volume in one second and forced vital capacity.

As used herein, the term “forced vital capacity” (FVC) refers to the vital capacity from a maximally forced expiratory effort, i.e., the total amount of air exhaled expelled by a subject during the FEV test.

As used herein, the term “FVC %” refers to the percent change in the FVC of a subject over a period of time. FVC % predicted is a subject's measured FVC expressed as the percentage of the predicted FVC for the subject. As used herein, all FVC % predicted values are absolute values and not relative value.

Many of these pulmonary function parameters are readily obtainable through the use of a spirometer. Residual volume can be obtained through indirect methods such as radiographic planimetry, body plethysmography, closed circuit dilution (including the helium dilution technique), and nitrogen washout.

Lung capacity and associated pulmonary function parameters naturally decline due to aging. Numerous studies have been conducted among normal populations to determine the rate of decline of lung capacity and other pulmonary function parameters. See Crapo et al. (1981) Am. Rev. Respir. Dis. 123:659-664. For example, a 65 years-old Caucasian male is expected to have a decline of 0.03 liters in FVC at age 66.

In contrast to the natural decline due to aging, subjects with lung disease such as pulmonary fibrosis and fibrosis caused by COVID-19 have an abnormally steep rate of decline in lung capacity or in one or more pulmonary function parameters, i.e., a “pathologic rate of decline.” As used herein, a “pathologic rate of decline” is a rate of decline in lung capacity or in one or more pulmonary function parameters that is at least 5% greater than the decline due to normal aging. In some embodiments, a pathologic rate of decline is at least 5%, 10%, 15%, 20%, 25%, 30%, 40%, 50%, 60%, 70%, 80%, 90%, 100%, 125%, 150%, 200%, 300%, 400%, 500%, 600%, 700%, 800% or 1000% greater than the predicted rate of decline for a normal person of similarly matched race or ethnicity, gender, age, height, and weight. Rates of decline can be expressed as the change from baseline per 1 week, 2 weeks, 4 weeks, 8 weeks, 12 weeks, 24 weeks, 36 weeks, 48 weeks, or 12 months. In particular embodiments, the pathologic rate of decline in lung capacity is the change in forced vital capacity (FVC) from baseline of at least about −0.05 liters, −0.10 liters, −0.15 liters, −0.20 liters or −0.25 liters per 12 months. In other embodiments, the pathological rate of decline is the change from baseline forced vital capacity percent (FVC %) predicted of at least about −2%, −3%, −4%, −5%, −6%, −7%, −8% or −10% per 12 months.

Administration of the ROCK2 inhibitors of the Formula I to XII may result in an increase of FVC in a subject with fibrosis after the treatment vs. before the treatment. Treatment with an effective amount of the ROCK2 inhibitor may increase FVC by at least 0.5%, 1%, 1.5%, 2.0%, 2.5%, 3.0%, 4.0%, 5.0%, 6.0%, 7.0%, 8.0%, 9.0%, 10%, 15%, 20%, 30%, 40% or 50% compared to FVC before the treatment. In some embodiments, treatment with ROCK2 inhibitor is for at least 1 week, 2 weeks, 3 weeks, 6 weeks, 9 weeks, 12 weeks, 15 weeks, 18 weeks, 21 weeks, 24 weeks, 27 weeks, 30 weeks, 33 weeks, 36 weeks or 48 weeks. In some embodiments, treatment is for 2 weeks or less, 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less from starting treatment with the ROCK2 inhibitor.

### Methods of Treating Arenaviridae Infections

In embodiments, the present application provides methods of treating Arenaviridae virus infection in a human, comprising: administering to the patient in need thereof, a therapeutically effective amount of a ROCK2 inhibitor disclosed herein, or a pharmaceutically acceptable salt, solvate, and/or ester thereof. In embodiments, and at least one additional active therapeutic agent is administered to the patient.

Also provided is the use of a ROCK2 inhibitor, or a pharmaceutically acceptable salt, solvate, and/or ester thereof, in the preparation of a medicament for use in treating an Arenaviridae infection in a human.

Arenaviridae are single-stranded negative sense RNA viruses that typically infect primates. Arenaviruses are able to multiply in virtually all cell types. Based upon studies in nonhuman primates infected with Lassa virus, the first cells infected appear to be dendritic cells in the lymphoid tissues. Infection progresses to infection of Kupffer cells in liver and parenchymal cells in liver and adrenal gland, endothelial cells in a variety of tissues including nervous tissue, and finally to infection of the epithelium. Evidence of liver infection in humans leading to hepatitis has also been documented) (Hensley, L., 2011, Virology Journal; Yun, N. E., 2012 Viruses).

There are 30 identified genera of Arenaviruses: Allpahuayo virus (ALLY), Amapari virus (AMAV), Bear Canyon virus (BCNV), Catarina virus, Chapare virus, Cupixi virus (CPXV), Dandenong virus, Flexal virus (FLEV), Guanarito virus (GTOV), Ippy virus (IPPYV), Junin virus (JUNV), Kodoko virus, Lassa virus (LASV; six strains—Josiah, NL, z148, Macenta, AV, and CSF), Latino virus (LATV), Lymphocytic choriomeningitis virus (LCMV), Lujo virus, Machupo virus (MACV), Mobala virus (MOBV), Morogoro virus, Mopeia virus (MOPV), Oliveros virus (OLVV), Parana virus (PARV), Pichinde virus (PICV), Pinhal virus, Pirital virus (PIRV), Sabia virus (SABV), Skinner Tank virus, Tacaribe virus (TCRV), Tamiami virus (TAMV), or Whitewater Arroyo virus (WWAV).

In some embodiments, the compound of the disclosure is administered in combination with an additional therapeutic agent. For the treatment of Arenaviridae virus infections, preferably, the additional therapeutic agent is active against Arenaviridae virus infections, particularly Lassa virus and Junin virus infections. Non-limiting examples of additional therapeutic agents include ribavirin, favipiravir (also known as T-705 or Avigan), T-705 monophosphate, T-705 diphosphate, T-705 triphosphate, ST-193, and mixtures thereof. The compounds and compositions of the present disclosure are also intended for use with general care provided patients with Arenaviridae viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

The compounds of this disclosure are useful in the treatment or prophylaxis of Arenaviridae infections in animals or in man.

However, in screening compounds capable of inhibiting Arenaviridae viruses, it should be kept in mind that the results of enzyme assays may not correlate with cell culture assays. Thus, a cell-based assay should be the primary screening tool.

In another aspect, the present application provides for methods of treating Arenaviridae virus infection in a human, comprising: administering to the patient a therapeutically effective amount of a ROCK2 inhibitor disclosed herein, or a pharmaceutically acceptable salt, solvate, and/or ester thereof. In some embodiments, the Arenaviridae infection is caused by an Arenaviridae virus. In some embodiments, the Arenaviridae infection is caused by a Junin virus. In some embodiments, the Arenaviridae infection is caused by Lassa virus strains Josiah, NL, z148, Macenta, AV, or CSF.

The compounds of the present disclosure can be used in the treatment of a human already suffering from an Arenaviridae infection or can be administered prophylactically to reduce or prevent the chance of an Arenaviridae infection. Physical examination of patients infected with arenavirus after the onset of fever often reveals purulent pharyngitis, bilateral conjunctival hemorrhages, facial edema, and generalized abdominal tenderness. Macroscopic pathological changes can include pleural effusions, pulmonary edema, ascites, and hemorrhagic manifestations in the gastrointestinal mucosa. Mortality rates for hospitalized patients vary between 5-10%.

### Kits

In one aspect, the present disclosure provides a kit or composition that includes a compound of Formulas I to XII, or a pharmaceutically acceptable salt, pharmaceutically acceptable ester, stereoisomer, mixture of stereoisomers or tautomer thereof. In separate embodiments individual kits are provided includes a compound of Formulas I to XII, or a pharmaceutically acceptable salt, pharmaceutically acceptable ester, stereoisomer, mixture of stereoisomers or tautomer thereof. In one aspect, the kit comprises a compound of Formulas I to XII, or a pharmaceutically acceptable salt thereof. Each of the individual kits described herein may comprise a label and/or instruction for use of the compound in the treatment of a disease or condition in a subject (e.g., human) in need thereof. In some embodiments, the disease or condition is a coronavirus infection. In some embodiments, the disease or condition is a human Arenaviridae viral infection, including a Lassa viral infection or a Junin viral infection. In other embodiments, each separate kit may also contain instructions for use of additional medical agents in combination with the compound of Formulas I to XII in the treatment of a disease or condition in a subject (e.g., human) in need thereof. In some embodiments, the kit comprises individual dose units of a compound described herein. Examples of individual dosage units may include pills, tablets, capsules, prefilled syringes or syringe cartridges, IV bags, etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit may contain a single dosage unit and in others multiple dosage units are present, such as the number of dosage units required for a specified regimen or period.

Also provided are articles of manufacture that include a compound of Formulas I to XII, or a pharmaceutically acceptable salt, pharmaceutically acceptable ester, stereoisomer, mixture of stereoisomers or tautomer thereof; and a container. In one aspect, the article of manufacture comprises a compound of Formulas I to XII, or a pharmaceutically acceptable salt thereof, and a container. In some embodiments, the container of the article of manufacture is a vial, jar, ampoule, preloaded syringe, blister package, tin, can, bottle, box, or an intravenous bag.

### EXAMPLES

### Example 1. In Vitro SARS-CoV-2 Prevention Model

A 50 mM stock solution of (6-(4-((4-(1H-pyrazol-4-yl)phenyl)amino)pyrimidin-2-yl)-1-methyl-1H-indol-2-yl)(3,3-difluoroazetidin-1-yl)methanone (Formula XII) in DMSO was diluted using PBS to Formula XII concentrations of 100 μM, 10 μM, 1 μM, 0.3 μM, 0.1 μM, and 0.03 μM. About 30,000 immortalized human airway epithelial cells (Calu-3) were grown to about 100,000 cells per well in a 96-well format. The cells were treated with the compound of Formula XII or control by adding 10 μl of a Formula XII solution (0.03-100 μM) or control solution (DMSO in PBS) in triplicate and incubating for 2 h at 37° C. The cells were then challenged with 10² PFU SARS-CoV-2 (2019-nCoV/USA-WA1/2020 strain), and incubated for 6 h at 37° C., followed by washing. A positive control consisted of alisporivir (5 μM). An additional control (3 wells) was treated with Formula XII at the highest concentration, but not challenged with virus. The cells were maintained until the endpoint at 48 h.

Viral infection was analyzed by qPCR (qRT-PCR). RNA from cells was extracted with TRIzol reagent (Thermo Fisher) according to manufacturer's instructions. Viral or host RNA levels in the supernatant were determined using the TaqPath™ 1-Step RT-qPCR Master Mix (Thermo Fisher) on CFX Connect Real-Time System (Bio-Rad) instrument using the following primers: Fwd: 5′-GACCCCAAAATCAGCGAAAT-3; Rev: 5′-TCTGGTTACTGCCAGTTGAATCTG-3′).

Cellular toxicity was measured by LDH (lactate dehydrogenase) assay (Clontech) according to the manufacturer's instructions. A positive control consisted of saponin (0.5%).

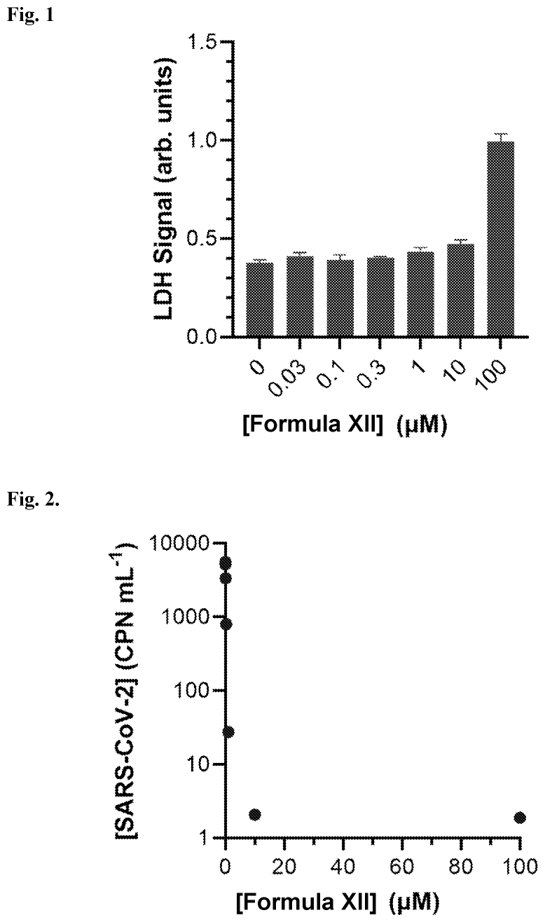

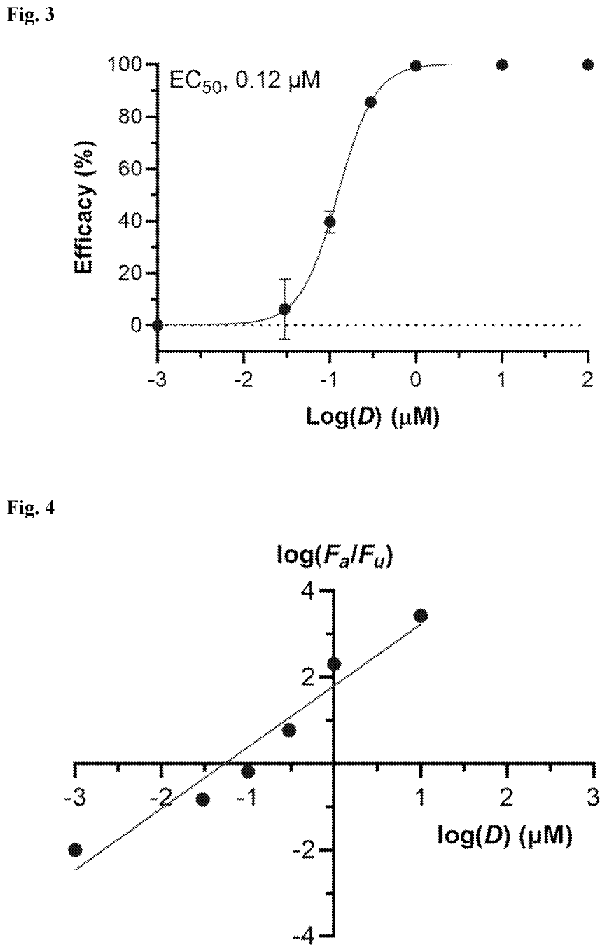

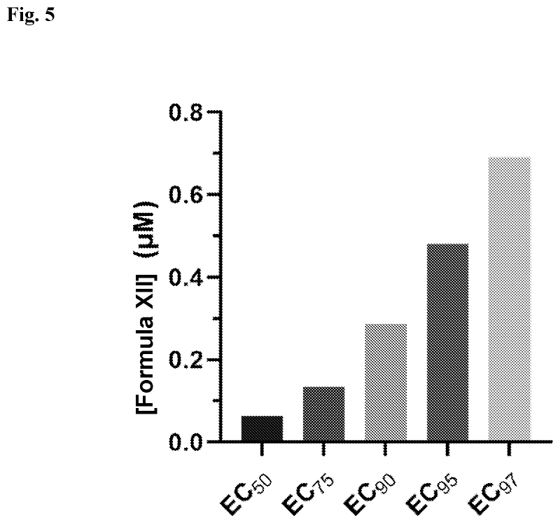

Datasets were analyzed and plotted in GraphPad Prism (version 9.0.2). Analytic simulations of dose-response curves using the median-effect principle and mass-action Law were carried out using CompuSyn (https://www.combosyn.com/). Results of the in vitro study are provided in FIGS. 1-5.

### Example 2: Formula XII Reduces the Spread of SARS-CoV-2 in the Lung in Vivo

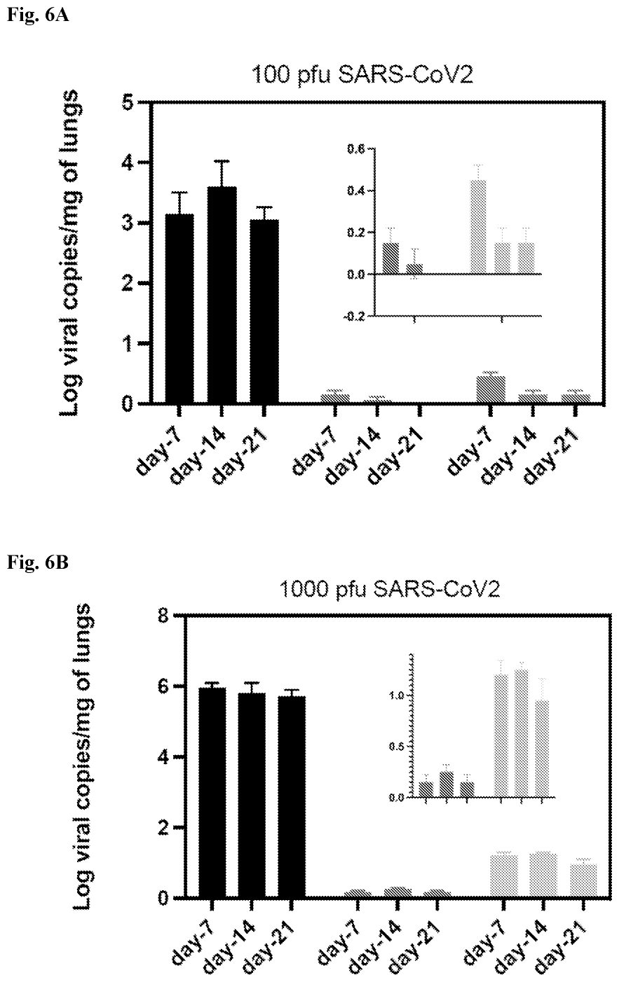

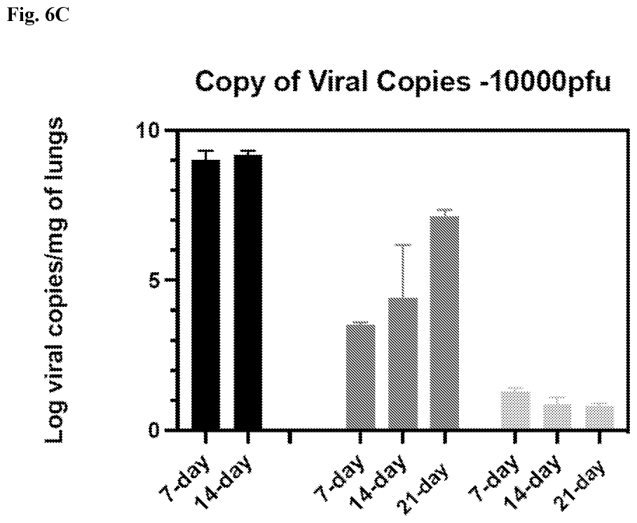

Human ACE2 transgenic mice from Jackson Laboratories (n=5 per group) were challenged at t=0 with SARS-CoV-2 at an initial viral load of 100 PFU, 1,000, or 10,000 pfu of 2019-nCoV/USA-WA1/2020 strain. This strain was isolated from an oropharyngeal swab from a patient with a respiratory illness who developed the clinical disease (COVID-19) in January 2020 in Washington, US (BEI Resources, NR-52281). A viral load of 10,000 pfu is considered to be significantly higher than the corresponding viral dose a human subject would occur in nature. The infected mice were dosed daily via oral gavage with (1) 0.5% sodium carboxymethyl cellulose (CMC-Na) (negative control), (2) 300 mg/kg Formula XII in 0.5% CMC-Na (test group), or (3) Remdesivir (RDV). RDV, an anti-viral reagent that acts as a nucleoside analog and inhibits the RNA-dependent RNA polymerase (RdRp) of coronaviruses including SARS-CoV-2, was used a positive control. RDV was solubilized at 2.5 mg/mL in vehicle containing 12% sulfobutylether-β-cyclodextrin sodium salt in water (with HCl/NaOH) at pH 5.0. (25 mg/kg subcutaneously) and continued every day until the end of the study. The animals were monitored daily. Two animals were sacrificed at the indicated times (FIG. 6). The viral load in the lung was determined by qPCR detecting viral RNA in lung lysates.

As shown in FIG. 6, treatment with Formula XII lead to a significantly reduced viral load as compared to both the negative as well as the positive control.

### Example 3. Treatment of COVID-19 Using ROCK2 Inhibitor

A study is conducted in men and women suspected of having or having SARS-CoV-2 infections. The study employs (6-(4-((4(4-(1H-pyrazol-4-yl)phenyl)amino)pyrimidin-2-yl)-1-methyl-1H-indol-2-yl)(3,3-difluoroazetidin-1-yl)methanone (Formula XII), which is orally administered 100-400 mg daily for 2-3 weeks of the treatment. Various amounts of Formula XII are administered at the designated intervals.

Subjects are observed from about two weeks to about six months. Coronavirus testing is conducted daily, every two days, every three days, or at other intervals during the treatment by collecting samples from a patient with a nasopharyngeal swab, an oropharyngeal swab, a nasal mid-turbinate swab, or an anterior nares swab. The swab is then placed immediately into a sterile transport tube containing about 2-3mL of either viral transport medium (VTM), Amies transport medium, or sterile saline, unless using a point-of-care test for coronaviruses. The sample is tested using a CDC authorized testing method. Different dosages of Formula XII administered 100 mg/day, 200 mg/day, 300 mg/day, or 400 mg/day are also tested in patients following the same protocols and record the dose-response relationship for the extent of virus inhibition.

### Example 4. Treatment of COVID-19 Using ROCK2 Inhibitor and Remdesivir

A study is conducted on men and women suspected of having, or having, SARS-CoV-2 infections. The study employs Formula XII, which is orally administered 100-400 mg daily for 2-3 weeks of the treatment. In parallel, remdesivir is administered via intravenous infusion in a total volume of up to 250 mL 0.9% saline over 30 to 120 minutes for adult patients according to the following schedule for adult and pediatric patients with body weight of at least 40 kg: on day 1, loading dose of 200 mg; on days 2-10, once-daily dose of about 100 mg.

Subjects are observed from about two weeks to about six months. Coronavirus testing is conducted daily, every two days, every three days, or at other intervals during the treatment by collecting samples from a patient with a nasopharyngeal swab, an oropharyngeal swab, a nasal mid-turbinate swab, or an anterior nares swab. The swab is then placed immediately into a sterile transport tube containing about 2-3 mL of either viral transport medium (VTM), Amies transport medium, or sterile saline, unless using a point-of-care test for coronaviruses. The sample is tested using a CDC authorized testing method. Different dosages of Formula XII administered 100 mg/day, 200 mg/day, 300 mg/day, and 400 mg/day are also tested in patients following the same protocols and the dose-response relationship is recorded for the extent of virus inhibition.

### Example 5. Treatment of Fibrosis Using ROCK2 Inhibitor

A study is conducted on men and women suspected of having, or having, SARS-CoV-2 infections and suspected of having, or having, fibrosis in the same way as described in Example 2. In addition to testing the extent of virus infection, respiratory symptoms, pulmonary function tests (PFTs), and/or high-resolution computed tomography (HRCT) are performed on the patient at selected intervals such as, for example, daily, every two, three, four, or five days, weekly, bi-weekly, or monthly, to monitor the progression of fibrosis. X-ray test and/or CT scans can be conducted if clinically approved. Further, the period of time over which Formula XII is administered can be extended in this study beyond two weeks at various dosage levels.

To assess pulmonary function of each of the subjects following treatment with FORMULA XII, the subjects are evaluated using a forced vital capacity (FVC) test. The Forced Expiratory Volume in One Second (FEV1), the amount of air that is forcefully exhaled in the first second of the FVC test, are measured for each subject either on day 1, day 3, day 7, day 14, monthly, or bi-monthly of the treatment.

### Example 6. Treatment of Fibrosis Using ROCK2 Inhibitor and Remdesivir

A study is conducted of 50 men and women suspected of having, or having, SARS-CoV-2 infections and suspected of having, or having, fibrosis in the same way as described in Example 2. In addition to testing the extent of virus infection, respiratory symptoms, pulmonary function tests (PFTs), and/or high-resolution computed tomography (HRCT) are performed on the patient at selected intervals such as, for example, daily, every two, three, four, or five days, weekly, bi-weekly, or monthly, to monitor the progression of fibrosis. X-ray test and/or CT scans can be conducted if clinically approved. Further, the period of time over which Formula XII is administered can be extended in this study beyond two weeks at various dosage levels.

To assess pulmonary function of each of the subjects following treatment with Formula XII, the subjects are evaluated using a forced vital capacity (FVC) test. The Forced Expiratory Volume in One Second (FEV1), the amount of air that is forcefully exhaled in the first second of the FVC test, are measured for each subject either on day 1, day 3, day 7, day 14, monthly, or bi-monthly of the treatment.

### Example 7. Treatment of Kawasaki-Like Disease Using ROCK2 Inhibitor

A study is conducted on pediatric patients suspected of having, or having, SARS-CoV-2 infections and suspected of having, or having, Kawasaki-like disease or displaying pediatric multisystem inflammatory syndrome (PMIS) or pediatric inflammatory multisystem syndrome temporally associated with SARS-CoV-2 (PIMS-TS). The study employs Formula XII, which is orally administered 100 mg/day, 200 mg/day, 300 mg/day, and 400 mg/day. Various amounts of Formula XII are administered at the designated intervals.

Subjects are observed from about two weeks to about six months. Coronavirus testing is conducted daily, every two days, every three days, or at other intervals during the treatment by collecting samples from a patient with a nasopharyngeal swab, an oropharyngeal swab, a nasal mid-turbinate swab, or an anterior nares swab. The swab is then placed immediately into a sterile transport tube containing about 2-3 mL of either viral transport medium (VTM), Amies transport medium, or sterile saline, unless using a point-of-care test for coronaviruses. The sample is tested using a CDC authorized testing method. Different dosages of Formula XII administered 50 mg/day, 100 mg/day, 150 mg/day, 200 mg/day, 250 mg/day, 300 mg/day, 350 mg/day, 400 mg/day, 450 mg/day, 500 mg/day, 550 mg/day, 600 mg/day, 650 mg/day, 700 mg/day, 750 mg/day, or 800 mg/day are also tested in patients following the same protocols and the dose-response relationship is recorded for the extent of virus inhibition.

In addition to testing the extent of virus infection, vasculitis tests, such as, for example, blood tests (for C-reactive protein, complete blood cell count, amounts of anti-neutrophil cytoplasmic antibodies, or other biomarkers), urine test (for red blood cells or protein contents), and/or imaging tests (e.g., X-rays, ultrasound, computerized tomography (CT), magnetic resonance imaging (MM) and positron emission tomography (PET)) are performed on the patient at selected intervals such as, for example, daily, every two, three, four, or five days, weekly, bi-weekly, or monthly, to monitor the progression of the Kawasaki-like disease, PMIS or PIMS-TS. Further, period of time over which Formula XII is administered can be extended in this study beyond two weeks at various dosage levels.

To assess the development of the disease, two-dimensional echocardiography is performed on patients to evaluate coronary artery lesions (CAL) at one month of illness. The measurement of each patient includes the diameter of the left main coronary artery (LMCA), the left anterior descending artery (LAD), the left circumflex coronary artery (LCX), and the proximal and middle segments of the right coronary artery (RCA). Z score of each coronary artery is calculated. See Journal of the American Society of Echocardiography, 2011, 24(1). CAL can be defined as z is least 2 of any coronary arteries of LMCA, LAD, LCX, and the proximal and middle segment of the RCA.

### Example 8. Treatment of Kawasaki-Like Disease Using ROCK2 Inhibitor and Remdesivir

A study is conducted of 20 pediatric patients suspected of having, or having, SARS-CoV-2 infections and suspected of having, or having, Kawasaki-like disease or displaying pediatric multisystem inflammatory syndrome (PMIS) or pediatric inflammatory multisystem syndrome temporally associated with SARS-CoV-2 (PIMS-TS). Formula XII, which is orally administered at 100-400 mg daily for 2-3 weeks of the treatment. Predetermined amounts of Formula XII are administered at designated intervals. In parallel for pediatric patients having a body weight between 3.5 kg and 40 kg, remdesivir is administered via intravenous infusion in a total volume of up to 125 mL 0.9% saline over 30 to 120 minutes: a single loading dose of 5 mg/kg on day 1; a daily loading dose of 2.5 mg/kg on days 2-10.

Subjects are observed from about two weeks to about six months. Coronavirus testing is conducted daily, every two days, every three days, or at other intervals during the treatment by collecting samples from a patient with a nasopharyngeal swab, an oropharyngeal swab, a nasal mid-turbinate swab, or an anterior nares swab. The swab is then placed immediately into a sterile transport tube containing about 2-3 mL of either viral transport medium (VTM), Amies transport medium, or sterile saline, unless using a point-of-care test for coronaviruses. The sample is tested using a CDC authorized testing method. Different dosages of Formula XII administered 50 mg/day, 100 mg/day, 150 mg/day, 200 mg/day, 250 mg/day, 300 mg/day, 350 mg/day, 400 mg/day, 450 mg/day, 500 mg/day, 550 mg/day, 600 mg/day, 650 mg/day, 700 mg/day, 750 mg/day, or 800 mg/day are also tested in patients following the same protocols and record the dose-response relationship for the extent of virus inhibition.

In addition to testing the extent of virus infection, vasculitis tests, such as, for example, blood tests (for C-reactive protein, complete blood cell count, amounts of anti-neutrophil cytoplasmic antibodies, or other biomarkers), urine test (for red blood cells or protein contents), and/or imaging tests (e.g., X-rays, ultrasound, computerized tomography (CT), magnetic resonance imaging (MM) and positron emission tomography (PET)) are performed on the patient at selected intervals such as, for example, daily, every two, three, four, or five days, weekly, bi-weekly, or monthly, to monitor the progression of the Kawasaki-like disease, PMIS or PIMS-TS. Further, the period of time over which Formula XII is administered can be extended in this study beyond two weeks at various dosage levels.

To assess the development of the disease, two-dimensional echocardiography is performed on patients to evaluate coronary artery lesions (CAL) at one month of illness. The measurement of each patient includes the diameter of the left main coronary artery (LMCA), the left anterior descending artery (LAD), the left circumflex coronary artery (LCX), and the proximal and middle segments of the right coronary artery (RCA). Z score of each coronary artery is calculated. See Renee Margossian et al., Journal of the American Society of Echocardiography, 2011, 24(1): 53-59. CAL can be defined as z is least 2 of any coronary arteries of LMCA, LAD, LCX, and the proximal and middle segment of the RCA.

1. A method for treating a viral infection in a patient in need thereof, the method comprising administering to the patient a therapeutically effective amount of a ROCK2 inhibitor having the Formula I: 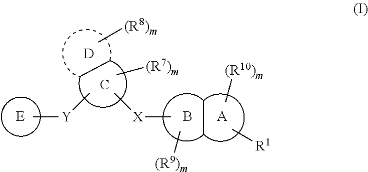
or a pharmaceutically acceptable salt, ester, stereoisomer, polymorph, solvate, N-oxide, or isotopically labeled compound thereof, wherein:
X and Y are each independently selected from the group consisting of a direct bond, C(═O), O, S(═O), and NR;
R is selected from the group consisting of H, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);
ring A and ring B are each independently selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O); provided that when ring B is a heterocycle containing a nitrogen atom, ring B is not attached to X via the nitrogen atom;
ring C is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring D is absent, or is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring E is selected from the group consisting of: 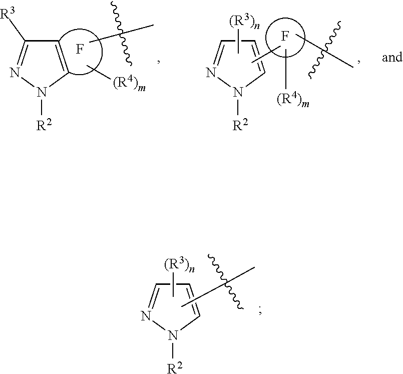
ring F is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
R¹ is selected from the group consisting of H, —NH₂, C₁₋₆ alkyl, C₆₋₁₀ aryl, 5- to 14-membered heteroaryl, N-methylpyrrolidinyl, N-methylpiperidinyl, 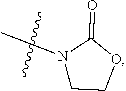 [SMILES: *N1CCOC1=O]
acetyl, 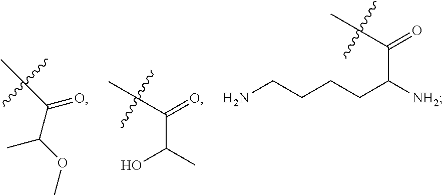
—C(═O)-(C₁₋₆ alkylene)n-CF₃, —C(═O)-(C₁₋₆ alkylene) CN, —C(═O)-(saturated or partially unsaturated C₃₋₁₀ cyclic hydrocarbyl), —NHC(═O)-(saturated or partially unsaturated C₃₋₁₀ cyclic hydrocarbyl), —C(═O)-(saturated or partially unsaturated 3- to 10-membered heterocyclyl), —C(═O)-C₁₋₆ alkylene-(saturated or partially unsaturated 3- to 10-membered heterocyclyl), —C(═O)-(5- to 14-membered heteroaryl), —C(═O)—C₁₋₆ alkylene-NH(C₁₋₆ alkyl), —C(═O)-C₁₋₆ alkylene-N(C₁₋₆ alkyl)₂, N-methylpiperazine substituted acetyl, —S(═O)₂R^{1a}, —P(═O)R^{1a}R^{1b}, 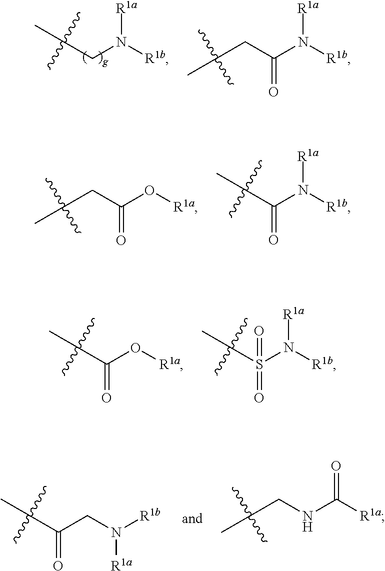
provided that when one of R¹ and R¹⁰ is C₁₋₆ alkyl, and the other is H or C₃₋₁₀ cyclic hydrocarbyl, at least one of X and Y is a direct bond, and ring C is not a 5-membered heteroaromatic ring; when one of R¹ and R¹⁰ is H, and the other is 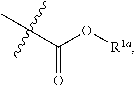
ring C is not a 5-membered heteroaromatic ring; when both R¹ and R¹⁰ are H, ring A contains at least one nitrogen atom, and is not a 5- or 6-membered ring; when one of R¹ and R¹⁰ is H, and the other is 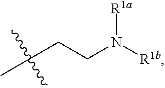
ring C is not a 5-membered heteroaromatic ring; and when one of R¹ and R¹⁰ is H, and the other is H or acetyl, ring D is absent;
R^{1a} and R^{1b} are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, NR⁵—S(═O)₂—R⁶, —NR⁵—C(═O)—NR⁵R⁶, -C₁₋₆ alkylene-NR⁵R⁶, -C₁₋₆ alkylene-OR⁵ and —O-C₁₋₆ alkylene-NR⁵R⁶, provided that when one of R^{1a} and R^{1b} is n-propyl, the other is not H; or R^{1a} and R^{1b} together with the atom to which they are attached form a 3- to 12-membered heterocycle or heteroaromatic ring;
R², R³, R⁴, R⁷, R⁸, R⁹ and R¹⁰, at each occurrence, are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, OC(═O)R⁵, —C(═O)OR⁵, OR⁵, SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, -C₁₋₆ alkylene-NR⁵R⁶, -C₁₋₆ alkylene-O(P═O)(OH)₂ and —O-C₁₋₆ alkylene-NR⁵R⁶;
the above alkyl, alkylene, alkenyl, alkynyl, cyclic hydrocarbyl, hydrocarbon ring, heterocyclyl, heterocycle, aryl, aromatic ring, heteroaryl, heteroaromatic ring and aralkyl, at each occurrence, are each optionally substituted with one or more substituents independently selected from the group consisting of halogen, hydroxyl, oxo, amino, cyano, nitro, C₁₋₆ alkyl, C₂₋₆ alkenyl, C₂₋₆ alkynyl, C₃₋₆ cyclic hydrocarbyl, 3- to 10-membered heterocyclyl, C₆₋₁₀ aryl, 5- to 14-membered heteroaryl, C₆₋₁₂ aralkyl, ═N—OR⁵, —C(═NH)NH₂, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, -C₁₋₆ alkylene-NR⁵R⁶ and —O-C₁₋₆ alkylene-NR⁵R⁶, and the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, C₁₋₆ alkyl, C₃₋₆ cyclic hydrocarbyl, 3- to 10-membered heterocyclyl, C₆₋₁₀ aryl, 5- to 14-membered heteroaryl and C₆₋₁₂ aralkyl;
R⁵ and R⁶, at each occurrence, are each independently selected from the group consisting of H, alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3;
n is an integer of 0, 1 or 2;
i is an integer of 0, 1 or 2; and
g is an integer of 0, 1, 2, 3 or 4.

2. The method of claim 1, wherein the ROCK2 inhibitor is a compound of formula II to IX: 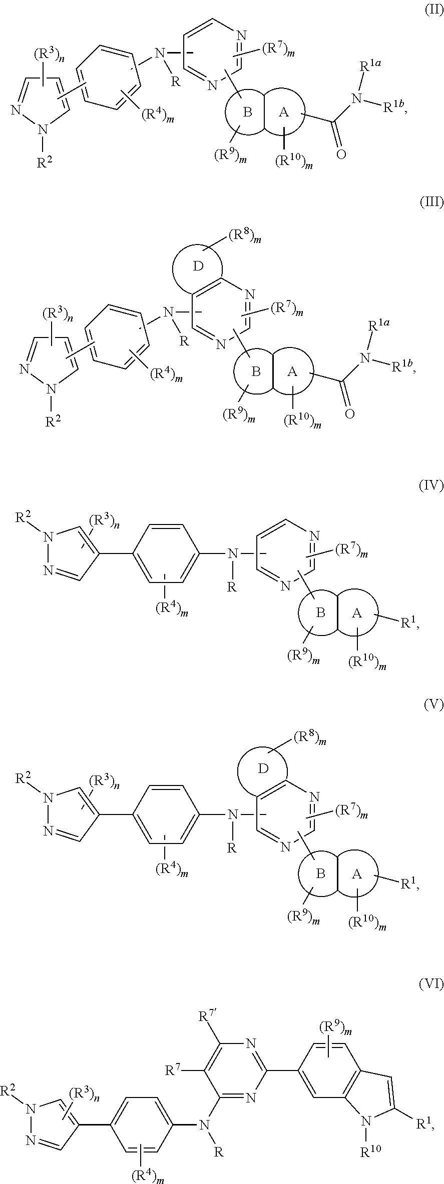 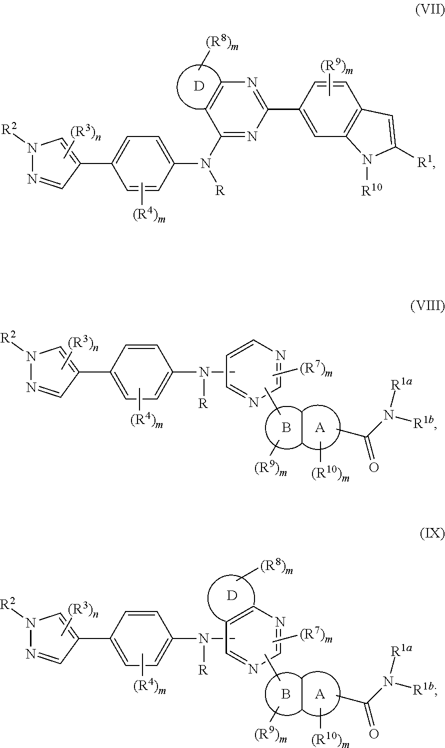
or a pharmaceutically acceptable salt, ester, stereoisomer, polymorph, solvate, N-oxide, or isotopically labeled compound thereof, wherein each of ring A, ring B, ring D, R, R¹, R^{1a}, R^{1b}, R², R³, R⁴, R⁷, R^{7′}, R⁸, R⁹, R¹⁰, n and m are defined above.

3. The method of claim 1, wherein the ROCK2 inhibitor is a compound of formula X or formula XI: 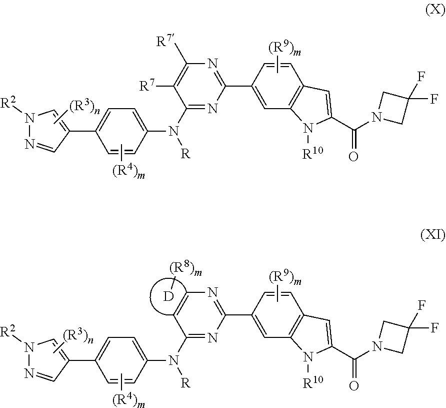
or a pharmaceutically acceptable salt, ester, stereoisomer, polymorph, solvate, N-oxide, or isotopically labeled compound thereof, wherein:
R is selected from the group consisting of H and C₁₋₆ alkyl;
ring D is saturated or partially unsaturated 3- to 10-membered heterocycle, C₆₋₁₀ aryl or 5- to 10-membered heteroaromatic ring;
R² is selected from the group consisting of H and C₁₋₆ alkyl;
R³, R⁴, R⁷, R^{7′} and R⁸, at each occurrence, are each independently selected from the group consisting of H, halogen, —NH₂, —OH, C₁₋₆ alkyl and —OR⁵;
R⁹ and R¹⁰, at each occurrence, are each independently selected from the group consisting of H, halogen, C₁₋₆ alkyl, C₂₋₆ alkenyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵ and -C₁₋₆ alkylene-O(P═O)(OH)₂;
the above alkyl, alkenyl, cyclic hydrocarbyl, heterocyclyl, aryl, heteroaryl, heteroaromatic ring and aralkyl, at each occurrence, are each optionally substituted with one or more substituents independently selected from the group consisting of halogen, C₁₋₆ alkyl and —OR⁵;
R⁵ and R⁶, at each occurrence, are each independently selected from the group consisting of H, C₁₋₆ alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3; and
n is an integer of 0, 1 or 2.

4. The method according to claim 1, wherein R⁵ and R⁶, at each occurrence, are each independently selected from the group consisting of H, methyl and ethyl.

5. The method according to claim 1, wherein R³, R⁴, R⁷, R^{7′}, and R⁸, at each occurrence, are each independently selected from the group consisting of H, F, Cl, Br, —NH₂, —OH, methyl, trifluoromethyl, —CH₂—Ph, methoxy, ethoxy and —CH₂OCH₃.

6. The method according to claim 1, wherein R⁹ and R¹⁰, at each occurrence, are each independently selected from the group consisting of H, F, Cl, Br, methyl, ethyl, n-propyl, isopropyl, vinyl, cyclopropyl, cyclobutyl, cyclopentyl, oxetanyl, monofluoromethyl, difluoromethyl, trifluoromethyl, acetyl, —OCH₂CHF₂, CH₂OH, —CH₂OCH₃, —CH₂CH₂OCH₃, —CH₂—O(P═O)(OH)₂,

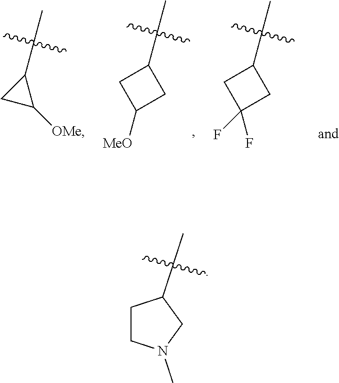

7. The method of claim 1, wherein the ROCK2 inhibitor is the compound (6-(4-((4-(1H-pyrazol-4-yl)phenyl)amino)pyrimidin-2-yl)-1-methyl-1H-indol-2-yl)(3,3-difluoroazetidin-1-yl)methanone having the chemical formula XII 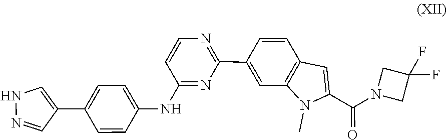 [SMILES: Cn1c(C(=O)N2CC(F)(F)C2)cc2ccc(-c3nccc(Nc4ccc(-c5cn[nH]c5)cc4)n3)cc21]
or a pharmaceutically acceptable salt, ester, stereoisomer, polymorph, solvate, N-oxide, or isotopically labeled compound thereof.

8. The method according to claim 1, wherein the viral infection is caused by a coronavirus.

9. A method for treating or preventing sequelae resulting from a viral infection in a patient in need thereof, the method comprising administering to the patient a therapeutically effective amount of a ROCK2 inhibitor having the Formula I: 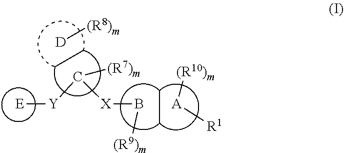
or a pharmaceutically acceptable salt, ester, stereoisomer, polymorph, solvate, N-oxide, or isotopically labeled compound thereof, wherein:
X and Y are each independently selected from the group consisting of a direct bond, C(═O), O, S(═O), and NR;
R is selected from the group consisting of H, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);
ring A and ring B are each independently selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O); provided that when ring B is a heterocycle containing a nitrogen atom, ring B is not attached to X via the nitrogen atom;
ring C is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring D is absent, or is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring E is selected from the group consisting of: 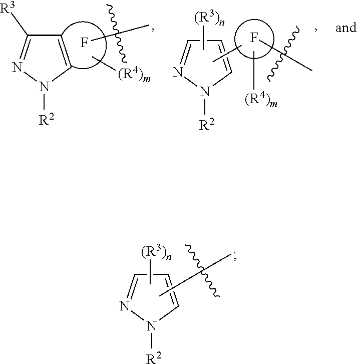
ring F is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
R¹ is selected from the group consisting of H, —NH₂, C₁₋₆ alkyl, C₆₋₁₀ aryl, 5- to 14-membered heteroaryl, N-methylpyrrolidinyl, N-methylpiperidinyl, 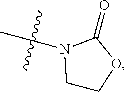 [SMILES: *N1CCOC1=O]
acetyl, 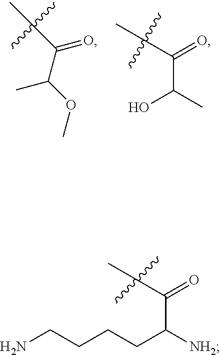
—C(═O)-(C₁₋₆ alkylene)n-CF₃, —C(═O)-(C₁₋₆ alkylene) CN, —C(═O)-(saturated or partially unsaturated C₃₋₁₀ cyclic hydrocarbyl), —NHC(═O)-(saturated or partially unsaturated C₃₋₁₀ cyclic hydrocarbyl), —C(═O)-(saturated or partially unsaturated 3- to 10-membered heterocyclyl), —C(═O)-C₁₋₆ alkylene-(saturated or partially unsaturated 3- to 10-membered heterocyclyl), —C(═O)-(5- to 14-membered heteroaryl), —C(═O)—C₁₋₆ alkylene-NH(C₁₋₆ alkyl), —C(═O)-C₁₋₆ alkylene-N(C₁₋₆ alkyl)₂, N-methylpiperazine substituted acetyl, —S(═O)₂R^{1a}, —P(═O)R^{1a}R^{1b}, 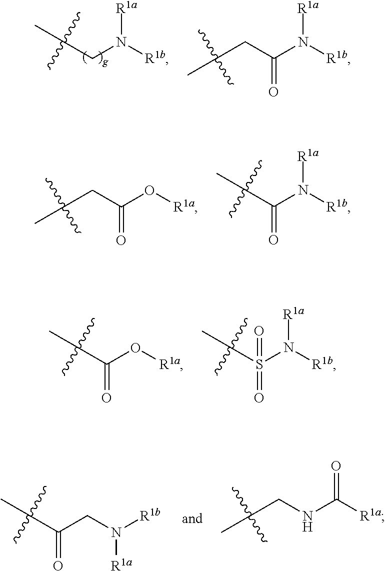
provided that when one of R¹ and R¹⁰ is C¹⁻⁶ alkyl, and the other is H or C³⁻¹⁰ cyclic hydrocarbyl, at least one of X and Y is a direct bond, and ring C is not a 5-membered heteroaromatic ring; when one of R¹ and R¹⁰ is H, and the other is 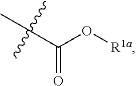
ring C is not a 5-membered heteroaromatic ring; when both R¹ and R¹⁰ are H, ring A contains at least one nitrogen atom, and is not a 5- or 6-membered ring; when one of R¹ and R¹⁰ is H, and the other is 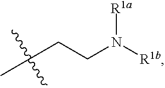
ring C is not a 5-membered heteroaromatic ring; and when one of R¹ and R¹⁰ is H, and the other is H or acetyl, ring D is absent;
R^{1a} and R^{1b} are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, NR⁵—S(═O)₂-R⁶, —NR⁵—C(═O)—NR⁵R⁶, -C₁₋₆ alkylene-NR⁵R⁶, -C₁₋₆ alkylene-OR⁵ and —O-C₁₋₆ alkylene-NR⁵R⁶, provided that when one of R^{1a} and R^{1b} is n-propyl, the other is not H; or R^{1a} and R^{1b} together with the atom to which they are attached form a 3- to 12-membered heterocycle or heteroaromatic ring;
R², R³, R⁴, R⁷, R⁸, R⁹ and R¹⁰, at each occurrence, are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, OC(═O)R⁵, —C(═O)OR⁵, OR⁵, SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, -C₁₋₆ alkylene-NR⁵R⁶, -C₁₋₆ alkylene-O(P═O)(OH)₂ and —O-C₁₋₆ alkylene-NR⁵R⁶;
the above alkyl, alkylene, alkenyl, alkynyl, cyclic hydrocarbyl, hydrocarbon ring, heterocyclyl, heterocycle, aryl, aromatic ring, heteroaryl, heteroaromatic ring and aralkyl, at each occurrence, are each optionally substituted with one or more substituents independently selected from the group consisting of halogen, hydroxyl, oxo, amino, cyano, nitro, C₁₋₆ alkyl, C₂₋₆ alkenyl, C₂₋₆ alkynyl, C₃₋₆ cyclic hydrocarbyl, 3- to 10-membered heterocyclyl, C₆₋₁₀ aryl, 5- to 14-membered heteroaryl, C₆₋₁₂ aralkyl, ═N—OR⁵, —C(═NH)NH₂, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, -C₁₋₆ alkylene-NR⁵R⁶ and —O-C₁₋₆ alkylene-NR⁵R⁶, and the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, C₁₋₆ alkyl, C₃₋₆ cyclic hydrocarbyl, 3- to 10-membered heterocyclyl, C₆₋₁₀ aryl, 5- to 14-membered heteroaryl and C₆₋₁₂ aralkyl;
R⁵ and R⁶, at each occurrence, are each independently selected from the group consisting of H, alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3;
n is an integer of 0, 1 or 2;
i is an integer of 0, 1 or 2; and
g is an integer of 0, 1, 2, 3 or 4.

10. The method of claim 9, wherein the ROCK2 inhibitor is a compound of formula II to IX: 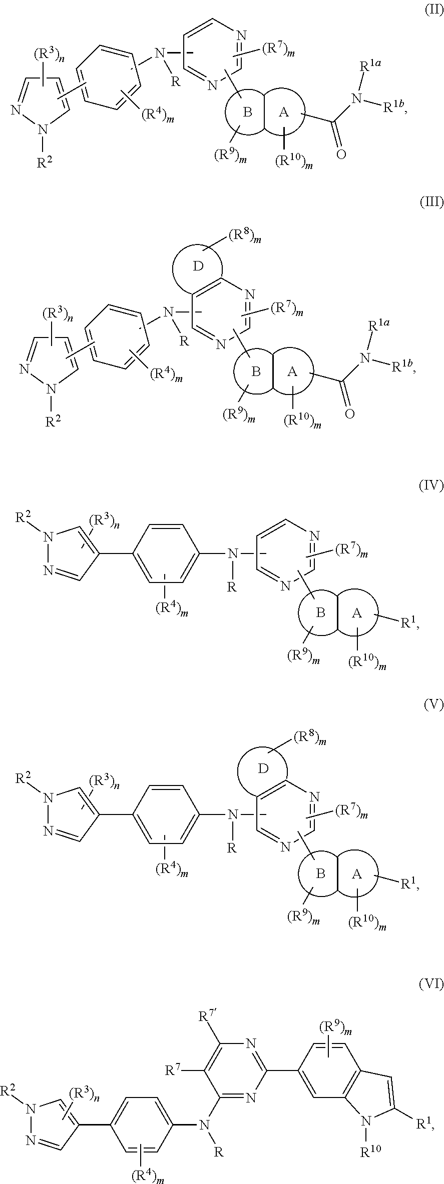 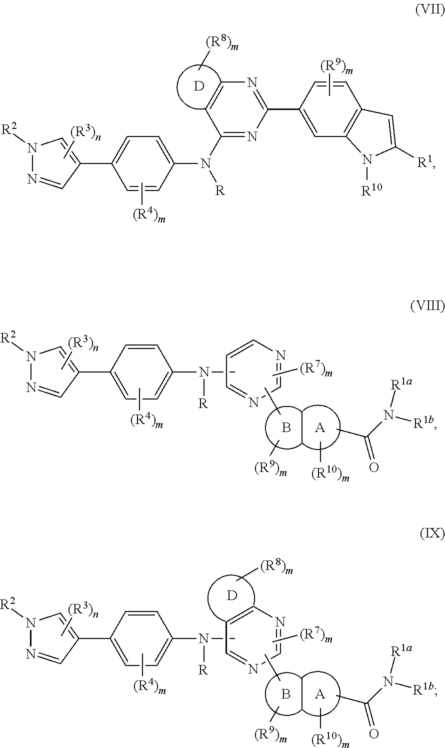
or a pharmaceutically acceptable salt, ester, stereoisomer, polymorph, solvate, N-oxide, or isotopically labeled compound thereof, wherein each of ring A, ring B, ring D, R, R¹, R^{1a}, R^{1b}, R², R³, R⁴, R⁷, R^{7′}, R⁸, R⁹, R¹⁰, n and m are defined above.

11. The method of claim 9, wherein the ROCK2 inhibitor is a compound of formula X or formula XI: 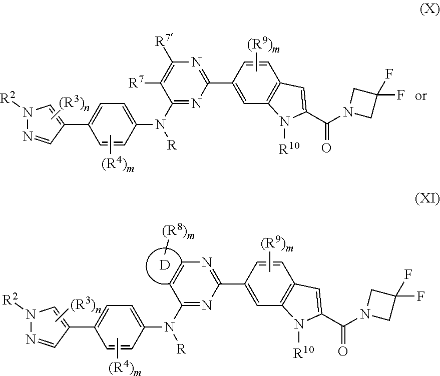
or a pharmaceutically acceptable salt, ester, stereoisomer, polymorph, solvate, N-oxide, or isotopically labeled compound thereof, wherein:
R is selected from the group consisting of H and C₁₋₆ alkyl;
ring D is saturated or partially unsaturated 3- to 10-membered heterocycle, C₆₋₁₀ aryl or 5- to 10-membered heteroaromatic ring;
R² is selected from the group consisting of H and C₁₋₆ alkyl;
R³, R⁴, R⁷, R^{7′} and R⁸, at each occurrence, are each independently selected from the group consisting of H, halogen, —NH₂, —OH, C₁₋₆ alkyl and —OR⁵;
R⁹ and R¹⁰, at each occurrence, are each independently selected from the group consisting of H, halogen, C₁₋₆ alkyl, C₂₋₆ alkenyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵ and -C₁₋₆ alkylene-O(P═O)(OH)₂;
the above alkyl, alkenyl, cyclic hydrocarbyl, heterocyclyl, aryl, heteroaryl, heteroaromatic ring and aralkyl, at each occurrence, are each optionally substituted with one or more substituents independently selected from the group consisting of halogen, C₁₋₆ alkyl and —OR⁵;
R⁵ and R⁶, at each occurrence, are each independently selected from the group consisting of H, C₁₋₆ alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3; and
n is an integer of 0, 1 or 2.

12. The method according to claim 9, wherein R⁵ and R⁶, at each occurrence, are each independently selected from the group consisting of H, methyl and ethyl.

13. The method according to claim 9 wherein R³, R⁴, R⁷, R^{7′} and R⁸, at each occurrence, are each independently selected from the group consisting of H, F, Cl, Br, —NH₂, —OH, methyl, trifluoromethyl, —CH₂—Ph, methoxy, ethoxy and —CH₂OCH₃.

14. The method according to claim 9 wherein R⁹ and R¹⁰, at each occurrence, are each independently selected from the group consisting of H, F, Cl, Br, methyl, ethyl, n-propyl, isopropyl, vinyl, cyclopropyl, cyclobutyl, cyclopentyl, oxetanyl, monofluoromethyl, difluoromethyl, trifluoromethyl, acetyl, —OCH₂CHF₂, CH₂OH, —CH₂OCH₃, —CH₂CH₂OCH₃, —CH₂—O(P═O)(OH)₂,

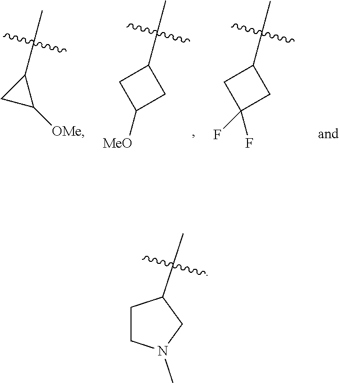

15. The method of claim 9, wherein the ROCK2 inhibitor has is the compound (6-(4-((4-(1H-pyrazol-4-yl)phenyl)amino)pyrimidin-2-yl)-1-methyl-1H-indol-2-yl)(3,3-difluoroazetidin-1-yl)methanone having the chemical formula XII 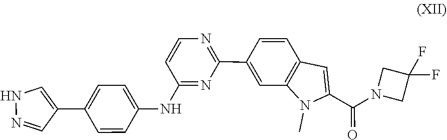 [SMILES: Cn1c(C(=O)N2CC(F)(F)C2)cc2ccc(-c3nccc(Nc4ccc(-c5cn[nH]c5)cc4)n3)cc21]
or a pharmaceutically acceptable salt, ester, stereoisomer, polymorph, solvate, N-oxide, or isotopically labeled compound thereof.

16. The method according to claim 1 wherein the viral infection is caused by a coronavirus.

17. The method according to claim 16, wherein the viral infection is caused by SARS-CoV-1, SARS-CoV-2 or MERS-CoV.

18. The method according to claim 17, wherein the viral infection is caused by SARS-CoV-2.

19. The method according to claim 18, wherein the viral infection is caused by SARS-CoV-2 variant Delta and/or SARS-CoV-2 variant Omicron.

20. The method according to claim 9 wherein the sequelae resulting from the viral infection include one or more of the group consisting of fatigue, dyspnea, cough, arthralgia, myalgia, headache, chest pain, fever, palpitations, myocardial inflammation, ventricular dysfunction, stroke, pulmonary function abnormalities, pulmonary fibrosis, renal dysfunction rash, alopecia, olfactory and/or gustatory dysfunction, sleep dysregulation, cognitive impairment altered, memory impairment, depression, anxiety, changes in mood, and combinations thereof.

21. The method according to claim 9 wherein the sequelae is fibrosis.

22. The method according to claim 1 wherein the ROCK2 inhibitor is administered to the patient at a total dose of about 200 mg to about 500 mg per day.

23. The method of claim 22, wherein the ROCK2 inhibitor is administered to the patient at a total dose of about 200 mg per day.

24. The method of claim 22, wherein the ROCK2 inhibitor is administered to the patient at a total dose of about 300 mg per day.

25. The method of claim 22, wherein the ROCK2 inhibitor is administered to the patient at a total dose of about 400 mg per day.

26. The method of claim 22, wherein the ROCK2 inhibitor is administered to the patient at a total dose of about 500 mg per day.

27. The method of claim 22, wherein the ROCK2 inhibitor is administered in one daily administration.

28. The method of claim 22, wherein the ROCK2 inhibitor is administered in two daily administrations.

29. The method of claim 1, wherein the ROCK2 inhibitor is administered to the patient within about 120 hours after the patient was first exposed to the virus causing the viral infection.

30. The method of claim 1, wherein the ROCK2 inhibitor is administered to the patient within about 120 hours after the patient has developed symptoms caused by the viral infection.

31. The method of claim 30, wherein the ROCK2 inhibitor is administered to the patient for at least about 1 week after the symptoms have subsided.
